SEQUENCE LISTING (100 sequences)

SEQ ID NO: 1 - protein, 14 aa, Artificial sequence
  Synthetic Polypeptide
FLFPLITSFLSKVL

SEQ ID NO: 2 - protein, 13 aa, Artificial sequence
  Synthetic Polypeptide
FISAIASMLGKFL

SEQ ID NO: 3 - protein, 13 aa, Artificial sequence
  Synthetic Polypeptide
GWFDVVKHIASAV

SEQ ID NO: 4 - protein, 13 aa, Artificial sequence
  Synthetic Polypeptide
FFGSVLKLIPKIL

SEQ ID NO: 5 - protein, 13 aa, Artificial sequence
  Synthetic Polypeptide
GLFDIIKKIAESF

SEQ ID NO: 6 - protein, 13 aa, Artificial sequence
  Synthetic Polypeptide
HGVSGHGQHGVHG

SEQ ID NO: 7 - protein, 13 aa, Artificial sequence
  Synthetic Polypeptide
FLPLIGRVLSGIL

SEQ ID NO: 8 - protein, 13 aa, Artificial sequence
  Synthetic Polypeptide
GLFDIIKKIAESI

SEQ ID NO: 9 - protein, 16 aa, Artificial sequence
  Synthetic Polypeptide
GLLDIVKKVVGAFGSL

SEQ ID NO: 10 - protein, 16 aa, Artificial sequence
  Synthetic Polypeptide
GLFDIVKKVVGALGSL

SEQ ID NO: 11 - protein, 16 aa, Artificial sequence
  Synthetic Polypeptide
GLFDIVKKVVGAIGSL

SEQ ID NO: 12 - protein, 16 aa, Artificial sequence
  Synthetic Polypeptide
GLFDIVKKVVGTLAGL

SEQ ID NO: 13 - protein, 16 aa, Artificial sequence
  Synthetic Polypeptide
GLFDIVKKVVGAFGSL

SEQ ID NO: 14 - protein, 16 aa, Artificial sequence
  Synthetic Polypeptide
GLFDIAKKVIGVIGSL

SEQ ID NO: 15 - protein, 17 aa, Artificial sequence
  Synthetic Polypeptide
GLFDIVKKIAGHIAGSI

SEQ ID NO: 16 - protein, 17 aa, Artificial sequence
  Synthetic Polypeptide
GLFDIVKKIAGHIASSI

SEQ ID NO: 17 - protein, 17 aa, Artificial sequence
  Synthetic Polypeptide
GLFDIVKKIAGHIVSSI

SEQ ID NO: 18 - protein, 13 aa, Artificial sequence
  Synthetic Polypeptide
FVQWFSKFLGRIL

SEQ ID NO: 19 - protein, 16 aa, Artificial sequence
  Synthetic Polypeptide
GLFDVIKKVASVIGGL

SEQ ID NO: 20 - protein, 16 aa, Artificial sequence
  Synthetic Polypeptide
GLFDIIKKVASVVGGL

SEQ ID NO: 21 - protein, 16 aa, Artificial sequence
  Synthetic Polypeptide
GLFDIIKKVASVIGGL

SEQ ID NO: 22 - protein, 15 aa, Artificial sequence
  Synthetic Polypeptide
VWPLGLVICKALKIC

SEQ ID NO: 23 - protein, 14 aa, Artificial sequence
  Synthetic Polypeptide
NFLGTLVNLAKKIL

SEQ ID NO: 24 - protein, 13 aa, Artificial sequence
  Synthetic Polypeptide
FLPLIGKILGTIL

SEQ ID NO: 25 - protein, 13 aa, Artificial sequence
  Synthetic Polypeptide
FLPIIAKVLSGLL

SEQ ID NO: 26 - protein, 13 aa, Artificial sequence
  Synthetic Polypeptide
FLPIVGKLLSGLL

SEQ ID NO: 27 - protein, 13 aa, Artificial sequence
  Synthetic Polypeptide
FLSSIGKILGNLL

SEQ ID NO: 28 - protein, 13 aa, Artificial sequence
  Synthetic Polypeptide
FLSGIVGMLGKLF

SEQ ID NO: 29 - protein, 9 aa, Artificial sequence
  Synthetic Polypeptide
TPFKLSLHL

SEQ ID NO: 30 - protein, 14 aa, Artificial sequence
  Synthetic Polypeptide
GILDAIKAIAKAAG

SEQ ID NO: 31 - protein, 12 aa, Artificial sequence
  Synthetic Polypeptide
LFDIIKKIAESF

SEQ ID NO: 32 - protein, 25 aa, Artificial sequence
  Synthetic Polypeptide
LFDIIKKIAESGFLFDIIKKIAESF

SEQ ID NO: 33 - protein, 24 aa, Artificial sequence
  Synthetic Polypeptide
GLLNGLALRLGKRALKKIIKRLCR

SEQ ID NO: 34 - protein, 14 aa, Artificial sequence
  Synthetic Polypeptide
GHHHHHHHHHHHHH

SEQ ID NO: 35 - protein, 10 aa, Artificial sequence
  Synthetic Polypeptide
FKCRRWQWRM

SEQ ID NO: 36 - protein, 10 aa, Artificial sequence
  Synthetic Polypeptide
KTCENLADTY

SEQ ID NO: 37 - protein, 13 aa, Artificial sequence
  Synthetic Polypeptide
ALFDIIKKIAESF

SEQ ID NO: 38 - protein, 13 aa, Artificial sequence
  Synthetic Polypeptide
GAFDIIKKIAESF

SEQ ID NO: 39 - protein, 13 aa, Artificial sequence
  Synthetic Polypeptide
GLADIIKKIAESF

SEQ ID NO: 40 - protein, 13 aa, Artificial sequence
  Synthetic Polypeptide
GLFAIIKKIAESF

SEQ ID NO: 41 - protein, 13 aa, Artificial sequence
  Synthetic Polypeptide
GLFDAIKKIAESF

SEQ ID NO: 42 - protein, 13 aa, Artificial sequence
  Synthetic Polypeptide
GLFDIAKKIAESF

SEQ ID NO: 43 - protein, 13 aa, Artificial sequence
  Synthetic Polypeptide
GLFDIIAKIAESF

SEQ ID NO: 44 - protein, 13 aa, Artificial sequence
  Synthetic Polypeptide
GLFDIIKAIAESF

SEQ ID NO: 45 - protein, 13 aa, Artificial sequence
  Synthetic Polypeptide
GLFDIIKKAAESF

SEQ ID NO: 46 - protein, 13 aa, Artificial sequence
  Synthetic Polypeptide
GLFDIIKKIAASF

SEQ ID NO: 47 - protein, 13 aa, Artificial sequence
  Synthetic Polypeptide
GLFDIIKKIAEAF

SEQ ID NO: 48 - protein, 13 aa, Artificial sequence
  Synthetic Polypeptide
GLFDIIKKIAESA

SEQ ID NO: 49 - protein, 13 aa, Artificial sequence
  Synthetic Polypeptide
GLFDIIHKIAESF

SEQ ID NO: 50 - protein, 13 aa, Artificial sequence
  Synthetic Polypeptide
GLFDIIKHIAESF

SEQ ID NO: 51 - protein, 13 aa, Artificial sequence
  Synthetic Polypeptide
GLFDIIKKIAHSF

SEQ ID NO: 52 - protein, 13 aa, Artificial sequence
  Synthetic Polypeptide
GLFDIIRKIAESF

SEQ ID NO: 53 - protein, 13 aa, Artificial sequence
  Synthetic Polypeptide
GLFDIIKRIAESF

SEQ ID NO: 54 - protein, 13 aa, Artificial sequence
  Synthetic Polypeptide
GLFDIIKKIARSF

SEQ ID NO: 55 - protein, 13 aa, Artificial sequence
  Synthetic Polypeptide
GLFDIIKKIADSF

SEQ ID NO: 56 - DNA, 39 bp, Artificial sequence
  Synthetic Polynucleotide
ggcctgtttgatattattaaaaaaattgcggaaagcttt

SEQ ID NO: 57 - DNA, 39 bp, Artificial sequence
  Synthetic Polynucleotide
gcgctgtttgatattattaaaaaaattgcggaaagcttt

SEQ ID NO: 58 - DNA, 39 bp, Artificial sequence
  Synthetic Polynucleotide
ggcgcgtttgatattattaaaaaaattgcggaaagcttt

SEQ ID NO: 59 - DNA, 39 bp, Artificial sequence
  Synthetic Polynucleotide
ggcctggcggatattattaaaaaaattgcggaaagcttt

SEQ ID NO: 60 - DNA, 39 bp, Artificial sequence
  Synthetic Polynucleotide
ggcctgtttgcgattattaaaaaaattgcggaaagcttt

SEQ ID NO: 61 - DNA, 39 bp, Artificial sequence
  Synthetic Polynucleotide
ggcctgtttgatgcgattaaaaaaattgcggaaagcttt

SEQ ID NO: 62 - DNA, 39 bp, Artificial sequence
  Synthetic Polynucleotide
ggcctgtttgatattgcgaaaaaaattgcggaaagcttt

SEQ ID NO: 63 - DNA, 39 bp, Artificial sequence
  Synthetic Polynucleotide
ggcctgtttgatattattgcgaaaattgcggaaagcttt

SEQ ID NO: 64 - DNA, 39 bp, Artificial sequence
  Synthetic Polynucleotide
ggcctgtttgatattattaaagcgattgcggaaagcttt

SEQ ID NO: 65 - DNA, 39 bp, Artificial sequence
  Synthetic Polynucleotide
ggcctgtttgatattattaaaaaagcggcggaaagcttt

SEQ ID NO: 66 - DNA, 39 bp, Artificial sequence
  Synthetic Polynucleotide
ggcctgtttgatattattaaaaaaattgcggcgagcttt

SEQ ID NO: 67 - DNA, 39 bp, Artificial sequence
  Synthetic Polynucleotide
ggcctgtttgatattattaaaaaaattgcggaagcgttt

SEQ ID NO: 68 - DNA, 39 bp, Artificial sequence
  Synthetic Polynucleotide
ggcctgtttgatattattaaaaaaattgcggaaagcgcg

SEQ ID NO: 69 - DNA, 39 bp, Artificial sequence
  Synthetic Polynucleotide
ggcctgtttgatattattcacaaaattgcggaaagcttt

SEQ ID NO: 70 - DNA, 39 bp, Artificial sequence
  Synthetic Polynucleotide
ggcctgtttgatattattaaacacattgcggaaagcttt

SEQ ID NO: 71 - DNA, 39 bp, Artificial sequence
  Synthetic Polynucleotide
ggcctgtttgatattattaaaaaaattgcgcacagcttt

SEQ ID NO: 72 - DNA, 39 bp, Artificial sequence
  Synthetic Polynucleotide
ggcctgtttgatattattcgcaaaattgcggaaagcttt

SEQ ID NO: 73 - DNA, 39 bp, Artificial sequence
  Synthetic Polynucleotide
ggcctgtttgatattattaaacgcattgcggaaagcttt

SEQ ID NO: 74 - DNA, 39 bp, Artificial sequence
  Synthetic Polynucleotide
ggcctgtttgatattattaaaaaaattgcgcgcagcttt

SEQ ID NO: 75 - DNA, 39 bp, Artificial sequence
  Synthetic Polynucleotide
ggcctgtttgatattattaaaaaaattgcggacagcttt

SEQ ID NO: 76 - protein, 640 aa, Artificial sequence
  Synthetic Polypeptide
MGGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGGSGGSGGSGGSGGSGGSGGSGGTASNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDGGS

SEQ ID NO: 77 - protein, 654 aa, Artificial sequence
  Synthetic Polypeptide
MGLFDIIKKIAESFASGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGGSGGSGGSGGSGGSGGSGGSGGTASNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDGGS

SEQ ID NO: 78 - protein, 657 aa, Artificial sequence
  Synthetic Polypeptide
MGLFDIIKKVASVIGGLASGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGGSGGSGGSGGSGGSGGSGGSGGTASNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDGGS

SEQ ID NO: 79 - protein, 669 aa, Artificial sequence
  Synthetic Polypeptide
MHHHHHHENLYFQGLFDIIKKVASVIGGLASGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGGSGGSGGSGGSGGSGGSGGSGGTASNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDGGS

SEQ ID NO: 80 - protein, 623 aa, Artificial sequence
  Synthetic Polypeptide
MGGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGGSGGSGGSGGSGGSGGSGGSGGSKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGGSHHHHHH

SEQ ID NO: 81 - protein, 637 aa, Artificial sequence
  Synthetic Polypeptide
MGLFDIIKKIAESFASGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGGSGGSGGSGGSGGSGGSGGSGGSKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGGSHHHHHH

SEQ ID NO: 82 - protein, 640 aa, Artificial sequence
  Synthetic Polypeptide
MGLFDIIKKVASVIGGLASGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGGSGGSGGSGGSGGSGGSGGSGGSKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEKGGSHHHHHH

SEQ ID NO: 83 - protein, 281 aa, Artificial sequence
  Synthetic Polypeptide
MGGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSLPETGHHHHHH

SEQ ID NO: 84 - protein, 307 aa, Artificial sequence
  Synthetic Polypeptide
MHHHHHHENLYFQGLFDIIKKIAESFASGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSLPETGHHHHHH

SEQ ID NO: 85 - protein, 281 aa, Artificial sequence
  Synthetic Polypeptide
MGGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGCGGSHHHHHH

SEQ ID NO: 86 - protein, 295 aa, Artificial sequence
  Synthetic Polypeptide
MGLFDIIKKIAESFASGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGCGGSHHHHHH

SEQ ID NO: 87 - protein, 298 aa, Artificial sequence
  Synthetic Polypeptide
MGLFDIIKKVASVIGGLASGGSGGSGGSGGSGGSGGSGGSGGSGGSSKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKTGGSGCGGSHHHHHH

SEQ ID NO: 88 - protein, 254 aa, Artificial sequence
  Synthetic Polypeptide
MGLNDIFEAQKIEWHEGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 89 - protein, 248 aa, Artificial sequence
  Synthetic Polypeptide
MGHHHHHHGGASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYK

SEQ ID NO: 90 - protein, 5 aa, Artificial sequence
  Synthetic Polypeptide
LPETG

SEQ ID NO: 91 - protein, 6 aa, Artificial sequence
  Synthetic Polypeptide
LPETGG

SEQ ID NO: 92 - protein, 3 aa, Artificial sequence
  Synthetic Polypeptide
GGG

SEQ ID NO: 93 - protein, 19 aa, Artificial sequence
  Synthetic Polypeptide
GLFDIIKKIAESFLPETGG

SEQ ID NO: 94 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 95 - protein, 5 aa, Artificial sequence
  Synthetic Polypeptide
LPESG

SEQ ID NO: 96 - protein, 5 aa, Artificial sequence
  Synthetic Polypeptide
LAETG

SEQ ID NO: 97 - protein, 5 aa, Artificial sequence
  Synthetic Polypeptide
LAESG

SEQ ID NO: 98 - protein, 7 aa, Artificial sequence
  Synthetic Polypeptide
LPETGGG

SEQ ID NO: 99 - protein, 6 aa, Artificial sequence
  Synthetic Polypeptide
ENLYFQ

SEQ ID NO: 100 - protein, 4 aa, Artificial Sequence
  Synthetic Polypeptide
GGGK

(19) United States
(12) Patent Application Publication
(10) Pub. No.: US 2018/0312542 A1
(43) Pub. Date: Nov. 1, 2018

(54) ENDOSOMAL ESCAPE PEPTIDES

(71) Applicant: President and Fellows of Harvard College, Cambridge, MA (US)

(72) Inventors: David R. Liu, Lexington, MA (US); Margie Li, Washington, DC (US); David B. Thompson, Cambridge, MA (US)

(73) Assignee: President and Fellows of Harvard College, Cambridge, MA (US)

(21) Appl. No.: 15/767,842

(22) PCT Filed: Oct. 19, 2016
(86) PCT No.: PCT/US16/57661

§ 371 Date: Apr. 12, 2018

Related U.S. Application Data
(60) Provisional application No. 62/244,018, filed on Oct. 20, 2015.

(51) Int. Cl. C07K 7/08; A61K 38/10; A61K 38/17; C07K 14/195; C07K 14/46; C07K 7/06; C12N 15/62; C07K 14/005; C07K 14/37; C07K 14/47

(52) U.S. Cl. CPC C07K 7/08; A61K 38/10; A61K 38/17; C07K 14/195; C07K 14/463; C07K 2319/50; C12N 15/62; C07K 14/005; C07K 14/37; C07K 14/4723; C07K 2319/10; C07K 7/06

(57) ABSTRACT

The inefficient delivery of proteins into mammalian cells remains a major barrier to realizing the therapeutic potential of many proteins. Previously, it has been demonstrated that superpositively charged proteins are efficiently endocytosed and can bring associated proteins and nucleic acids into cells. The vast majority of cargo delivered in this manner, however, remains in endosomes and does not reach the cytosol. The present invention provides endosomal escape peptides that enhance endosomal escape and cytosolic delivery of proteins and other agents of interest. In one aspect, described herein are novel fusion proteins comprising endosomal escape peptides fused to proteins and other agents of interest for delivery to a cell. Also provided herein are methods and compounds useful in preparing the fusion proteins, as well as pharmaceutical compositions and uses of the fusion proteins.

### RELATED APPLICATIONS

This application claims priority under 35 U.S.C. § 119(e) to U.S. provisional patent application, U.S. Ser. No. 62/244,018, filed Oct. 20, 2015, which is incorporated herein by reference.

### GOVERNMENT SUPPORT

This invention was made with government support under grant numbers R01 GM095501 and R01 DC006908 awarded by the National Institutes of Health. The government has certain rights in the invention.

### BACKGROUND OF THE INVENTION

Proteins that bind extracellular targets, including monoclonal antibodies, Fc fusions, and cytokines, have served as important therapeutics. See, e.g., Nelson, et al. Nat Rev Drug Discov 2010, 9, 767; Huang, C. Current opinion in biotechnology 2009, 20, 692; Hafler, D. A. Nat Rev Immunol 2007, 7, 423; Leader et al. Nat Rev Drug Discov 2008, 7, 21. Fully realizing the therapeutic potential of proteins, however, requires methods to enable exogenous proteins to access intracellular targets. Because the vast majority of proteins cannot spontaneously cross cell membranes, the development of intracellular protein delivery methods could facilitate applications including enzyme replacement therapies for metabolic diseases, transcription factor-driven changes in cell fate, and genome editing. See, e.g., Schiffmann et al. JAMA 2001, 285, 2743; Spiegelman, B. M. Diabetes 1998, 47, 507; Mali, P.; Esvelt, K. M.; Church, G. M. Nat Meth 2013, 10, 957. Several methods for protein delivery have been explored in the past decade, including cell-penetrating peptides (CPPs), penta-arg proteins, receptor ligands, and lipid nanoparticles. While these and other methods have advanced the field of protein delivery, challenges including cytotoxicity, lack of generality, low potency, or poor in vivo activity continue to limit their therapeutic relevance. See, e.g., Mueller et al. Bioconjugate Chemistry 2008, 19, 2363; Appelbaum et al. Chemistry & Biology 2012, 19, 819; Rizk et al. Proceedings of the National Academy of Sciences 2009, 106, 11011; Hasadsri et al. Journal of Biological Chemistry 2009, 284, 6972; Fu et al. Bioconjugate Chemistry 2014, 25, 1602; Pisal et al. Journal of Pharmaceutical Sciences 2010, 99, 2557.

Superpositively charged proteins, a class of engineered and naturally occurring proteins that have abnormally high net positive charge, are known for their ability to potently deliver proteins and nucleic acids into mammalian cells. See, e.g., McNaughton et al. Proceedings of the National Academy of Sciences 2009, 106, 6111; Cronican et al. ACS Chemical Biology 2010, 5, 747; Cronican et al. Chemistry & Biology 2011, 18, 833; Lawrence et al. Journal of the American Chemical Society 2007, 129, 10110; International Patent Application Nos.: PCT/US2007/070254, PCT/US2009/041984, and PCT/US2010/001250. While superpositively charged proteins are very efficiently endocytosed and can be more effective for protein delivery than CPPs, the vast majority of endocytosed proteins remain sequestered in endosomes that either mature into lysosomes, resulting in protein degradation, or are recycled to the surface of the cell, resulting in extracellular protein release (FIG. 1A). As a result, relatively high concentrations (μM) of exogenous protein are typically needed for modest cytosolic or nuclear delivery. Although superpositively charged proteins can slow endosomal maturation, the inefficiency of endosomal escape enables only a small fraction of delivered protein to reach the cytosol. See, e.g., Thompson et al. Chemistry & Biology 2012, 19, 831; Fuchs et al. ACS Chemical Biology 2007, 2, 167; Pirie et al. Journal of Biological Chemistry 2011, 286, 4165; Varkouhi et al. Journal of Controlled Release 2011, 151, 220.

To address this protein delivery bottleneck, new peptides that facilitate endosomal escape when fused to endocytosed proteins are of great interest. Membrane-active peptides such as influenza-derived HA2 have been reported to be endosomolytic. See, e.g., Wadia et al. Nat Med 2004, 10, 310. However, many of these peptides, including HA2, are cytotoxic at concentrations required for protein delivery. See, e.g., Neundorf et al. Pharmaceuticals 2009, 2, 49; Sugita et al. British Journal of Pharmacology 2008, 153, 1143. In light of the foregoing, there remains a great need for new peptides that promote endosomal escape of proteins and other molecules.

### SUMMARY OF THE INVENTION

Because the vast majority of proteins cannot spontaneously cross cell membranes, the development of intracellular protein delivery systems, compositions, and methods could facilitate applications including enzyme replacement therapies for metabolic diseases, transcription factor-driven changes in cell fate, and genome editing. While certain classes of proteins (e.g., superpositively charged proteins) are efficiently endocytosed, the vast majority of endocytosed proteins remain sequestered in endosomes. A major challenge to intracellular protein delivery remains in promoting the endosomal escape and cytosolic delivery of proteins and other agents of interest. Described herein are peptide sequences which, when fused to proteins and other agents of interest, help facilitate ensodomal escape.

In one aspect, the present invention provides novel fusion proteins comprising a peptide, which promotes endosomal escape (referred to herein as “endosomal escape peptide” or “endosomal escape peptide sequence”), fused to a protein. The endosomal escape peptide can aid in cytosolic delivery of the protein. In certain embodiments, the novel fusion proteins of the present invention comprise an endosomal escape peptide fused to protein that aids in cellular delivery (e.g., a superpositively charged protein). In certain embodiments, the fusion protein comprises an endosomal escape peptide, a protein that aids in cellular delivery (e.g., a superpositively charged protein), and one or more additional agents to be delivered (e.g., proteins, peptides) to a cell. In some instances, the fusion proteins of the present invention exhibit greater levels of cytosolic delivery when compared to analogous proteins which lack the endosomal escape peptides described herein.

In another aspect, the present invention provides novel conjugates comprising an endosomal escape peptide fused to an agent (e.g., small molecule, peptide, or nucleic acid) for cellular delivery. The endosomal escape peptide can aid in cytosolic delivery of the agent. In certain embodiments, the conjugate comprises an endosomal escape peptide fused to a small molecule (i.e., a therapeutic small molecule or small molecule drug). In other embodiments, the conjugate comprises an endosomal escape peptide fused to a nucleic acid (e.g., DNA, RNA, or a hybrid thereof). Conjugates of the present invention may further comprise additional agents (e.g., proteins, peptides, nucleic acids, small molecules) for delivery to a cell.

The present invention also provides methods, compositions, systems, reagents, kits, and compounds useful in the preparation of the fusion proteins and conjugates described herein. Fusion proteins of the present invention can be assembled by conjugating an endosomal escape peptide to a protein. Likewise, conjugates of the present invention can be assembled by conjugating an endosomal escape peptide to an agent comprising nucleic acid or small molecule. Any method for conjugation or ligation known in the art may be used to conjugate an endosomal escape peptide to a protein or other agent of interest to form a fusion protein or conjugate of the present invention. Exemplary methods include, but are not limited to, amide/peptide bond-forming reactions, click chemistry reactions, and other bioconjugation techniques; see, e.g., Stephanopoulos et al. Nature Chemical Biology 2011, 7(12): 876-884. In one aspect, the present invention provides methods for the preparation of fusion proteins and conjugates that are based on a sortase-mediated ligation. In general, this method comprises contacting a substrate of the structure, [first peptide]-[first sortase recognition motif], with a substrate of the structure, [second sortase recognition motif]-[second agent], in the presence a sortase.

Fusion proteins and conjugates of the present invention can be assembled by conjugating an endosomal escape peptide to sortase recognition motif, forming a substrate of structure: [first peptide]-[first sortase recognition motif], which is then ligated to a protein or other agent of interest. Any reactions known in the art can be used to conjugate the endosomal escape peptide to a sortase recognition motif (e.g., peptide/amide bond-forming reactions, click chemistry reactions).

Fusion proteins of the present invention can be assembled by conjugating a sortase recognition motif to a protein, to form a substrate of structure: [second sortase recognition motif]-[second agent], which is then ligated to an endosomal escape peptide. Any method known in the art can be used to conjugate the protein of interest to the sortase recognition motif. Conjugates of the present invention can be assembled by conjugating a sortase recognition motif to an agent comprising a small molecule or nucleic acid, to form a substrate of structure: [second sortase recognition motif]-[second agent], which is then ligated to an endosomal escape peptide. Any method known in the art can be used to conjugate the small molecule or nucleic acid of interest to the sortase recognition motif.

In another aspect, the present invention provides novel peptides/reagents which are useful in the preparation of the fusion proteins and conjugates described herein. In general, these novel peptides are of the structure: [first peptide]-[first sortase recognition motif], wherein the “first peptide” is a endosomal escape peptide described herein, and the “first sortase recognition motif” is any handle for sortase ligation that is known in the art. In some embodiments, the “first sortase recognition motif” comprises an LPXT sequence, wherein X is any amino acid (e.g., LPETG (SEQ ID NO: 90); LPETGG (SEQ ID NO: 91)). In another aspect, the present invention provides novel peptides which have been shown to promote endosomal escape and cytosolic delivery when fused to proteins and other agents.

In another aspect, the present invention provides pharmaceutical compositions of the fusion proteins and conjugates described herein. The invention also provides methods for administering to a subject the fusion proteins and conjugates described herein. In yet another aspect, the present invention provides kits comprising any of the fusion proteins described herein, or pharmaceutical compositions thereof.

The details of certain embodiments of the invention are set forth in the Detailed Description of Certain Embodiments, as described below. Other features, objects, and advantages of the invention will be apparent from the Definitions, Examples, Figures, and Claims.

### Definitions

As used herein, the terms “fused,” “conjugated,” “ligated” or “attached,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. Two moieties may be physically associated with each other via covalent or non-covalent interactions, or a combination thereof. In some embodiments, a sufficient number of weaker interactions can provide sufficient stability for moieties to remain physically associated under a variety of different conditions.

As used herein, the term “protein” refers to a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., addition of a chemical entity such as a carbohydrate group, a phosphate group, a farnesyl group, an isofarnesyl group, a fatty acid group, an amide group, a terminal acetyl group, a linker for conjugation, functionalization, or other modification (e.g., alpha amidation), etc. In another embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. In certain embodiments, the modifications of the peptide lead to a more biologically active peptide. In some embodiments, polypeptides may comprise natural amino acids, non-natural amino acids, synthetic amino acids, amino acid analogs, and combinations thereof. The term “peptide” is typically used to refer to a polypeptide having a length of less than about 50 amino acids.

The term “fusion protein” refers to a protein comprising a plurality of heterologous proteins, protein domains, or peptides, e.g., a peptide fused to a supercharged protein fused to a third agent.

As used herein, the term “supercharged” refers to any protein with a modification that results in the increase or decrease of the overall net charge of the protein when compared with the parent protein. “Superpositively charged” refers to an increase in the overall net charge. Modifications include, but are not limited to, alterations in amino acid sequence or addition of charged moieties (e.g., carboxylic acid groups, phosphate groups, sulfate groups, amino groups). Supercharged proteins may be naturally occurring (i.e., wild-type) or syntherically modified. Examples of naturally occurring supercharged proteins contemplated as being within the scope of the present include, but are not limited to, cyclon, PNRC1, RNPS1, SURF6, AR6P, NKAP, EBP2, LSM11, RL4, KRR1, RY-1, BriX, MNDA, H1b, cyclin, MDK, Midkine, PROK, FGFS, SFRS, AKIP, CDK, beta-defensin, Defensin 3, PAVAC, PACAP, eotaxin-3, histone H2A, HMGB1, C-Jun, TERF 1, N-DEK, PIAS 1, Ku70, HBEGF, and HGF. In certain embodiments, the supercharged protein utilized in the invention is U4/U6.U5 tri-snRNP-associated protein 3, beta-defensin, Protein SFRS121P1, midkine, C—C motif chemokine 26, surfeit locus protein 6, Aurora kinase A-interacting protein, NF-kappa-B-activating protein, histone H1.5, histone H2A type 3, 60S ribosomal protein L4, isoform 1 of RNA-binding protein with serine-rich domain 1, isoform 4 of cyclin-dependent kinase inhibitor 2A, isoform 1 of prokineticin-2, isoform 1 of ADP-ribosylation factor-like protein 6-interacting protein 4, isoform long of fibroblast growth factor 5, or isoform 1 of cyclin-L1. For other examples of supercharged proteins contemplated in the present invention, including other examples of superpositively charged green fluorescent proteins, see International Patent Application Nos.: PCT/US2007/070254, PCT/US2009/041984, and PCT/US2010/001250; all of which are incorporated herein by reference.

As used herein, the term “green fluorescent protein” (GFP) refers to a protein originally isolated from the jellyfish Aequorea victoria that fluoresces green when exposed to blue light or a derivative of such a protein (e.g., a supercharged version of the protein). The amino acid sequence of wild type GFP is as follows:

(SEQ ID NO: 94)
MSKGEELFTG VVPILVELDG DVNGHKFSVS GEGEGDATYG
KLTLKFICTT GKLPVPWPTL VTTFSYGVQC FSRYPDHMKQ
HDFFKSAMPE GYVQERTIFF KDDGNYKTRA EVKFEGDTLV
NRIELKGIDF KEDGNILGHK LEYNYNSHNV YIMADKQKNG
IKVNFKIRHN IEDGSVQLAD HYQQNTPIGD GPVLLPDNHY
LSTQSALSKD PNEKRDHMVL LEFVTAAGIT HGMDELYK.

Proteins that are at least 70%, 75%, 80%, 85%, 90%, 95%, 98%, or 99% homologous are also considered to be green fluorescent proteins. In certain embodiments, the green fluorescent protein is supercharged. In certain embodiments, the green fluorescent protein is super positively charged (e.g., +36 GFP, as described herein). In certain embodiments, the GFP may be modified to include a polyhistidine tag for ease in purification of the protein. In certain embodiments, the GFP may be fused with another protein or peptide. In certain embodiments, the GFP may be further modified biologically or chemically (e.g., post-translational modifications, proteolysis, etc.).

The term “amino acid” refers to a molecule containing both an amino group and a carboxyl group. Amino acids include alpha-amino acids and beta-amino acids, the structures of which are depicted below. In certain embodiments, an amino acid is an alpha amino acid.

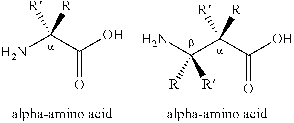

Suitable amino acids include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V, as provided below), unnatural alpha-amino acids natural beta-amino acids (e.g., beta-alanine), and unnnatural beta-amino acids. Exemplary natural alpha-amino acids include L-Alanine (A), L-Arginine (R), L-Asparagine (N), L-Aspartic acid (D), L-Cysteine (C), L-Glutamic acid (E), L-Glutamine (Q), Glycine (G), L-Histidine (H), L-Isoleucine (I), L-Leucine (L), L-Lysine (K), L-Methionine (M), L-Phenylalanine (F), L-Proline (P), L-Serine (S), L-Threonine (T), L-Tryptophan (W), L-Tyro sine (Y), and L-Valine (V). Exemplary unnatural alpha-amino acids include D-Arginine, D-Asparagine, D-Aspartic acid, D-Cysteine, D-Glutamic acid, D-Glutamine, D-Histidine, D-Isoleucine, D-Leucine, D-Lysine, D-Methionine, D-Phenylalanine, D-Proline, D-Serine, D-Threonine, D-Tryptophan, D-Tyrosine, D-Valine, Di-vinyl, α-methyl-Alanine (Aib), α-methyl-Arginine, α-methyl-Asparagine, α-methyl-Aspartic acid, α-methyl-Cysteine, α-methyl-Glutamic acid, α-methyl-Glutamine, α-methyl-Histidine, α-methyl-Isoleucine, α-methyl-Leucine, α-methyl-Lysine, α-methyl-Methionine, α-methyl-Phenylalanine, α-methyl-Proline, α-methyl-Serine, α-methyl-Threonine, α-methyl-Tryptophan, α-methyl-Tyrosine, α-methyl-Valine, Norleucine, terminally unsaturated alpha-amino acids and bis alpha-amino acids (e.g., modified cysteine, modified lysine, modified tryptophan, modified serine, modified threonine, modified proline, modified histidine, modified alanine, and the like). There are many known unnatural amino acids any of which may be included in the peptides of the present invention. See for example, S. Hunt, The Non-Protein Amino Acids: In Chemistry and Biochemistry of the Amino Acids, edited by G. C. Barrett, Chapman and Hall, 1985.

As used herein, the term “nucleic acid” refers to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, i.e., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications' A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In general, a “small molecule” refers to a non-peptidic, non-oligomeric organic compound either prepared in the laboratory or found in nature. Small molecules, as used herein, can refer to compounds that are “natural product-like;” however, the term “small molecule” is not limited to “natural product-like” compounds. Rather, a small molecule is typically characterized in that it contains several carbon-carbon bonds, and has a molecular weight of less than 1500 g/mol, less than 1250 g/mol, less than 1000 g/mol, less than 750 g/mol, less than 500 g/mol, or less than 250 g/mol, although this characterization is not intended to be limiting for the purposes of the present invention. In certain other embodiments, natural-product-like small molecules are utilized.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe).

As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

The term “sortase,” as used herein, refers to a protein having sortase activity, i.e., an enzyme able to carry out a transpeptidation reaction conjugating the C-terminus of a protein to the N-terminus of a protein via transamidation. The term includes full-length sortase proteins, e.g., full-length naturally occurring sortase proteins, fragments of such sortase proteins that have sortase activity, modified (e.g., mutated) variants or derivatives of such sortase proteins or fragments thereof, as well as proteins that are not derived from a naturally occurring sortase protein, but exhibit sortase activity. Those of skill in the art will readily be able to determine whether or not a given protein or protein fragment exhibits sortase activity, e.g., by contacting the protein or protein fragment in question with a suitable sortase substrate under conditions allowing transpeptidation and determining whether the respective transpeptidation reaction product is formed. In some embodiments, a sortase is a protein comprising at least 20 amino acid residues, at least 30 amino acid residues, at least 40 amino acid residues, at least 50 amino acid residues, at least 60 amino acid residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues. In some embodiments, a sortase is a protein comprising less than 100 amino acid residues, less than 125 amino acid residues, less than 150 amino acid residues, less than 175 amino acid residues, less than 200 amino acid residues, or less than 250 amino acid residues. Non-limiting examples of sortases that can be used in the disclosed methods are described herein and additional suitable sortases will be apparent to those of skill in the art. For example, in some embodiments, a sortase is employed that comprises an amino acid sequence that is at least 90% homologous to the amino acid sequence of wild-type S. aureus Sortase A or a fragment thereof having sortase activity, e.g., a fragment comprising at least amino acids 61-206 of wild-type S. aureus Sortase A. In some embodiments, a mutant sortase is employed. Typically, the mutant sortase exhibits enhanced reaction kinetics as compared to wild type sortase, e.g., a higher reaction efficiency or a higher reaction rate. Mutant sortases that are suitable are described elsewhere herein, and include, for example, sortases comprising one or more mutations selected from the group consisting of P94S, P94R, E106G, F122Y, F154R, D160N, D165A, G174S, K190E, and K196T.

Typically, a “sortase” utilizes two substrates: (1) a substrate comprising a C-terminal “sortase recognition motif”; and (2) a second substrate comprising an N-terminal “sortase recognition motif”; and the transpeptidation reaction results in a conjugation of both substrates via a covalent bond. Some sortase recognition motifs are described herein and additional suitable sortase recognition motifs are well known to those of skill in the art. For example, sortase A of S. aureus recognizes and utilizes a C-terminal LPXT motif, wherein X is any amino acid) and an N-terminal GGG (SEQ ID NO: 92) motif in transpeptidation reactions. Additional sortase recognition motifs will be apparent to those of skill in the art, and the invention is not limited in this respect. A sortase substrate may comprise an LPXT motif, the N-terminus of which is conjugated to any agent, e.g., a peptide, protein, a small molecule, nucleic acid. Similarly, a sortase substrate may comprise a GGG motif, the C-terminus of which is conjugated to any agent, e.g., a peptide, protein, a small molecule, nucleic acid.

As generally defined herein, “click chemistry” or “click chemsitry reaction” is any covalent bond-forming reaction which may be used to join two molecules. Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition). 60: 384-395. Exemplary coupling reactions (some of which may be classified as “Click

### BRIEF DESCRIPTION OF THE DRAWINGS

The accompanying drawings, which constitute a part of this specification, illustrate several embodiments of the invention and together with the description, serve to explain the principles of the invention.

FIGS. 1A-1B. (FIG. 1A) Overview of protein delivery in mammalian cells. Cationic macromolecules such as +36 GFP interact with anionic sulfated proteoglycans on the cell surface and are endocytosed and sequestered in early endosomes. The early endosomes can acidify into late endosomes or lysosomes. Alternatively, early endosomes may be trafficked back to the cell surface as part of the membrane-recycling pathway. To access the cytoplasm, an exogenous cationic protein must escape endosomes before it is degraded or exported. (FIG. 1B) Sortase-mediated conjugation of peptides with +36 GFP-Cre recombinase prior to screening. Sortase was used to conjugate synthetic peptides containing a C-terminal LPETGG (SEQ ID NO: 91) with expressed+36 GFP-Cre containing an N-terminal GGG. The resulting peptide-LPETGGG (SEQ ID NO: 98)-+36 GFP-Cre fusion proteins have the same chemical composition as expressed recombinant proteins but are more easily assembled.

FIG. 2. Primary screen for cytosolic delivery of Cre recombinase in BSR.LNL.tdTomato cells. Initial screen of 20 peptide-(+36 GFP)-Cre conjugated proteins. Cytosolic Cre delivery results in recombination and tdTomato expression. The percentage of tdTomato positive cells was determined by fluorescence image analysis. 250 nM+36 GFP-Cre was used as the no-peptide control (NP), and addition of 100 μM chloroquine was used as the positive control (+). Cells were treated with 250 nM protein for 4 hours in serum-free DMEM. Cells were washed and supplanted with full DMEM and incubated for 48 hours. Error bars represent the standard deviation of three independent biological replicates.

FIGS. 3A-3B. Efficacy and toxicity of recombinant expression fusions of aurein 1.2 (“E”) and citropin 1.3 (“U”). (FIG. 3A) Cytosolic Cre delivery results in recombination and tdTomato expression. The percentage of tdTomato positive cells was determined by flow cytometry. Protein fusions were delivered at 125 nM, 250 nM, 500 nM, and 1 μM. (FIG. 3B) Toxicity of aurein 1.2 and citropin 1.3 as determined by CellTiterGlo (Promega) assay. Protein fusions were delivered at 125 nM, 250 nM, 500 nM, and 1 μM. The labeled concentration of +36 GFP-Cre was used as the no peptide control (NP), and addition of 100 μM chloroquine was used as the positive control (+). Cells were treated with 250 nM protein for 4 hours in serum-free media. Cells were washed and supplanted with full DMEM and incubated for 48 hours. Error bars represent the standard deviation of three independent biological replicates.

FIGS. 4A-4B. Activity and cytotoxicity of aurein 1.2 variants fused to +36 GFP-Cre. (FIG. 4A) The percentage of tdTomato positive cells was determined by flow cytometry. (FIG. 4B) Toxicity as determined by CellTiterGlo (Promega) assay. For FIG. 4A and FIG. 4B, 250 nM+36 GFP-Cre was used as the no peptide control (NP), and addition of 100 μM chloroquine was used as the positive control (+). Cells were treated with 250 nM protein for 4 hours in serum-free DMEM. Cells were washed and supplanted with full DMEM and incubated for 48 hours.

FIGS. 5A-5D. Investigating the ability of +36 GFP and aurein 1.2-+36 GFP dexamethasone-conjugates to reach the cytosol and activate GR translocation. (FIG. 5A) Images of HeLa cells expressing GR-mCherry treated in the presence and absence of 1 μM dexamethasone (Dex)-protein conjugates for 30 minutes at 37° C. (FIG. 5B) Nuclear-to-cytosol GR-mCherry fluorescence ratios (translocation ratios) of respective Dex-protein conjugates determined using CellProfiler®. (FIG. 5C) GR-mCherry translocation ratios resulting from cells treated in the presence and absence of +36 GFP^{Dex} and endocytic inhibitors. (FIG. 5D) GR-mCherry translocation ratios resulting from cells treated in the presence and absence of aurein 1.2-+36 GFP^{Dex} and endocytic inhibitors. Statistical significance is measured by P-value. ns=P>0.05, *=P≤0.05, **=P≤0.01, ***=P≤0.001.

FIGS. 6A-6C. In vivo protein delivery of Cre recombinase into mouse neonatal cochleas. 0.4 μL of 50 μM+36 GFP-Cre or aurein 1.2-+36 GFP-Cre were injected into the scala media. (FIG. 6A) Five days after injection, cochlea were harvested. Inner hair cells (IHC), outer hair cells (OHC) and supporting cells in the sensory epithelium (SE) were imaged for the presence of tdTomato, which is only expressed following Cre-mediated recombination. Hair cells were labeled with antibodies against the hair-cell marker Myo7a. (FIG. 6B) To evaluate cytotoxicity, the number of outer hair cells and inner hair cells were measured by counting DAPI-stained cells. (FIG. 6C) The percentage of tdTomato positive cells, reflecting successful delivery of functional Cre recombinase, was determined by fluorescence imaging.

FIGS. 7A-7C. Representative mass spectra of evolved sortase-mediated conjugation reactions of peptide-LPETGG (SEQ ID NO: 91) to GGG-+36GFP-Cre. Three spectra were chosen as examples to demonstrate all observed scenarios: multiple conjugation products (FIG. 7A), one conjugation product (FIG. 7B), and no conjugation (FIG. 7C). Conjugation efficiency was determined through LC-MS using protein deconvolution through MaxEnt (Waters) by comparing relative peak intensities. Multiple conjugation products are possible for peptides that begin with an N-terminal glycine, since those peptides can act as a nucleophile for the sortase reaction to generate oligomeric peptides. In such cases, expression and purification of full-length protein fusions is helpful to characterize the activity of single species.

FIGS. 8A-8B. Cre-mediated recombination assay in BSR.LNL.tdTomato cells. (FIG. 8A) Fluorescence imaging analysis of treated cells where percent recombination was determined by dividing the number of TRITC (tdTomato) positive cells by the number of DAPI (Hoesct-treated) positive cells. (FIG. 8B) Flow cytometry analysis of treated cells where percent recombination was determined by gating for PE-A (tdTomato) cells out of the total cell population after forward and side scatter gating.

FIG. 9. Determining the delivery efficiency of aurein 1.2 in trans with +36 GFP-Cre. 125 nM, 250 nM, or 500 nM+36 GFP-Cre was mixed with either aurein 1.2-+36 GFP (125 nM, 250 nM, 500 nM) or with aurein 1.2 (1 μM, 10 μM, 100 μM), then assayed for Cre-mediated recombination as measured by tdTomato signal during flow cytometry. Addition of 100 μM chloroquine was used as a positive control. The expressed fusion aurein 1.2-+36 GFP-Cre protein at 125 nM, 250 nM, or 500 nM was used as the positive control.

FIGS. 10A-10C. Evolved sortase-mediated conjugation of GGGK^{Dex} (SEQ ID NO: 100) to +36 GFP-LPETGG (SEQ ID NO: 91) and aurein 1.2-+36 GFP-LPETGG (SEQ ID NO: 91). (FIG. 10A) Mass spectra to GGGK^{Dex} (SEQ ID NO: 100). (FIG. 10B) Coomassie gel of unreacted and reacted+36 GFP-LPETGG (SEQ ID NO: 91) and aurein 1.2-+36 GFP-LPETGG (SEQ ID NO: 91). (FIG. 10C) Western blot of unreacted and reacted+36 GFP-LPETGG (SEQ ID NO: 91) and aurein 1.2-+36 GFP-LPETGG (SEQ ID NO: 91). Fluorescent signal detected by anti-dexamethasone antibody.

FIGS. 11A-11B. Analysis of +36 GFP-BirA and aurein 1.2-+36 GFP-BirA delivery. (FIG. 11A) Western blot images of biotin and mCherry signal from Li-COR IRdye antibodies. Biotin signal is proportional to the amount of BirA delivered into the cytosol. mCherry-AP was transfected into HeLa cells and used as a transfection and loading control. (FIG. 11B) Quantitative biotin signal was determined by normalizing the raw biotin signal to the raw mCherry signal. 100 μM chloroquine with 250 nM+36 GFP-BirA was used as a positive control.

FIGS. 12A-12D. In vivo delivery of +36 GFP-Cre, aurein 1.2-+36 GFP-Cre, and citropin 1.3-+36 GFP-Cre. (FIGS. 12A-12B) Toxicity as determined by observed number of cells. (FIGS. 12C-12D) Percent tdTomato-positive (recombined) cells as determined directly by fluorescence imaging.

FIG. 13. Preparation of dexamethasone-21-thiopropionic Acid (SDex) for labeling peptide amines on solid phase. Inset shows analytical HPLC trace of SDex.

FIGS. 14A-14D. Cytosolic fractionation to quantify non-endosomal and total cellular protein delivery. (FIG. 14A)+36 GFP or aurein 1.2-+36 GFP protein at 250 nM, 500 nM, or 1 μM was incubated with HeLa cells for 30 min in serum-free media, then washed and resuspended in isotonic sucrose (290 mM sucrose, 10 mM imidazole, pH 7.0 with 1 mM DTT and cOmplete EDTA-free protease inhibitor cocktail), homogenized, and pelleted at 350,000 g for 30 minutes. The fluorescence of the supernatant (cytosolic fraction) was analyzed on a fluorescence plate reader and compared to that of standard curves (FIGS. 14B-14C) relating fluorescence to known concentrations of +36 GFP and aurein 1.2-+36-GFP. (FIG. 14D) Total cellular protein delivery was measured by incubating+36 GFP or aurein 1.2-+36 GFP protein at 250 nM, 500 nM, or 1 μM with HeLa cells for 30 min in serum-free media. Cells were washed three times with PBS containing 20 U/mL heparin to remove surface-bound protein, then pelleted, washed with PBS, and pelleted at 500 g for 3 minutes. Flow cytometry of the resulting cells revealed the total amount of delivered protein. Error bars represent the standard deviation of three separate aliquots of cytosolic extract. Statistical significance is measured by P-value (ns=P>0.05, *=P≤0.05, **=P≤0.01, ***=P≤0.001).

### DETAILED DESCRIPTION OF CERTAIN EMBODIMENTS

Promoting endosomal escape is a challenge in the delivery of agents to intracellular targets. The present invention provides systems, compounds, compositions, reagents, and related methods and uses for enhancing endosomal escape and cytosolic delivery of proteins and other agents to cells. As described herein, cytosolic delivery of a protein or other agent of interest (e.g., peptide, nucleic acid, small molecule) can be promoted by associating the protein or other agent with an endosomal escape peptide sequence as described herein. In one aspect, the prevent invention provides novel fusion proteins comprising at least one protein fused to an endosomal escape peptide sequence. In some embodiments of the present invention, the fusion proteins comprise a superpositively charged protein for promoting cellular delivery (e.g., a green fluorescent protein) and an endosomal escape peptide sequence for promoting endosomal escape, and a third agent (e.g., peptide, protein) for delivery to the cell. In another aspect, provided herein are conjugates comprising an endosomal escape peptide fused to an agent (e.g., small molecule, peptide, nucleic acid) for delivery to a cell. In general, these fusion proteins and conjugates exhibit a greater propensity for cytosolic delivery as compared with proteins and other agents which lack one of the endosomal escape peptide sequences described herein. The fusion proteins and conjugates described herein, or compositions thereof, can be administered to cells in vitro or in vivo.

The present invention also provides methods for preparing fusion proteins and conjugates comprising endosomal escape peptides, and intermediates in the preparation thereof. As described herein, any method for conjugation or ligation known in the art (e.g., peptide/amide bond forming reactions, click chemistry reactions) can be used to conjugate an endosomal escape peptide to a protein or other agent of interest. In some embodiments, the method for preparing a fusion protein or conjugate of the present invention involves a sortase-mediated ligation. Also provided herein are novel peptides and proteins which are useful as building blocks in the assembly of novel fusion proteins (e.g., via sortase-mediated ligation). In some instances, assembly of the fusion protein via sortase-mediated ligation is more efficient than recombinant expression of the fusion proteins. In general, the systems, compounds, compositions, reagents, kits, and related methods and uses for delivery of proteins and other agents provided herein exhibit improved efficacy, reduced cytotoxicity, and/or ease of preparation as compared to current celluar delivery technologies.

### Fusion Proteins

The present invention provides novel fusion proteins comprising a endosomal escape peptide sequence fused to a protein for delivery to a cell. The endosomal escape peptide sequence promote endocomal escape and cytosolic delivery of the protein. In certain embodiments, the fusion protein comprises a peptide sequence (referred to herein as “endosomal escape peptide” or “endosomal escape peptide sequence”) that is at least 90% identical to any one of the amino acid sequences set forth in SEQ ID NOs 1-55 (see Table 1, Table 2, or Table A) fused to a protein for delivery to a cell.

TABLE A

| SEQ ID NO: | Amino Acid Sequence |
|---|---|
| 1 | FLFPLITSFLSKVL |
| 2 | FISAIASMLGKFL |
| 3 | GWFDVVKHIASAV |
| 4 | FFGSVLKLIPKIL |
| 5 | GLFDIIKKIAESF |
| 6 | HGVSGHGQHGVHG |
| 7 | FLPLIGRVLSGIL |
| 8 | GLFDIIKKIAESI |
| 9 | GLLDIVKKVVGAFGSL |
| 10 | GLFDIVKKVVGALGSL |
| 11 | GLFDIVKKVVGAIGSL |
| 12 | GLFDIVKKVVGTLAGL |
| 13 | GLFDIVKKVVGAFGSL |
| 14 | GLFDIAKKVIGVIGSL |
| 15 | GLFDIVKKIAGHIAGSI |
| 16 | GLFDIVKKIAGHIASSI |
| 17 | GLFDIVKKIAGHIVSSI |
| 18 | FVQWFSKFLGRIL |
| 19 | GLFDVIKKVASVIGGL |
| 20 | GLFDIIKKVASVVGGL |
| 21 | GLFDIIKKVASVIGGL |
| 22 | VWPLGLVICKALKIC |
| 23 | NFLGTLVNLAKKIL |
| 24 | FLPLIGKILGTIL |
| 25 | FLPIIAKVLSGLL |
| 26 | FLPIVGKLLSGLL |
| 27 | FLSSIGKILGNLL |
| 28 | FLSGIVGMLGKLF |
| 29 | TPFKLSLHL |
| 30 | GILDAIKAIAKAAG |
| 31 | LFDIIKKIAESF |
| 32 | LFDIIKKIAESGFLFDIIKKIAESF |
| 33 | GLLNGLALRLGKRALKKIIKRLCR |
| 34 | GHHHHHHHHHHHHH |
| 35 | FKCRRWQWRM |
| 36 | KTCENLADTY |
| 37 | ALFDIIKKIAESF |
| 38 | GAFDIIKKIAESF |
| 39 | GLADIIKKIAESF |
| 40 | GLFAIIKKIAESF |
| 41 | GLFDAIKKIAESF |
| 42 | GLFDIAKKIAESF |
| 43 | GLFDIIAKIAESF |
| 47 | GLFDIIKAIAESF |
| 45 | GLFDIIKKAAESF |
| 46 | GLFDIIKKIAASF |
| 47 | GLFDIIKKIAEAF |
| 48 | GLFDIIKKIAESA |
| 59 | GLFDIIHKIAESF |
| 50 | GLFDIIKHIAESF |
| 51 | GLFDIIKKIAHSF |
| 52 | GLFDIIRKIAESF |
| 53 | GLFDIIKRIAESF |
| 54 | GLFDIIKKIARSF |
| 55 | GLFDIIKKIADSF |

In certain embodiments, the endosomal escape peptide sequence is at least 95% identical to any one of the amino acid sequences set forth in SEQ ID NOs: 1-55. In certain embodiments, the endosomal escape peptide sequence is at least 98% identical to any one of the amino acid sequences set forth in SEQ ID NOs: 1-55. In certain embodiments, the endosomal escape peptide sequence is at least 99% identical to any one of the amino acid sequences set forth in SEQ ID NOs: 1-55. In certain embodiments, the endosomal escape peptide sequence is identical to any one of the amino acid sequences set forth in SEQ ID NOs: 1-55. In certain embodiments, the endosomal escape peptide sequence is at least 90% identical to the amino acid sequence set forth in SEQ ID NO: 5. In certain embodiments, the endosomal escape peptide sequence is at least 95% identical to the amino acid sequence set forth in SEQ ID NO: 5. In certain embodiments, the endosomal escape peptide sequence is at least 98% identical to the amino acid sequence set forth in SEQ ID NO: 5. In certain embodiments, the endosomal escape peptide sequence is at least 99% identical to the amino acid sequence set forth in SEQ ID NO: 5. In certain embodiments, the endosomal escape peptide sequence is identical to the amino acid sequences set forth in SEQ ID NO: 5. In certain embodiments, the endosomal escape peptide sequence comprises any one of the amino acid sequences set forth in SEQ ID NOs: 1-55; optionally with 1, 2, 3, 4, or 5 amino acid additions, substitutions, deletions, mutations, or any combination thereof. In certain embodiments, the endosomal escape peptide sequence comprises the amino acid sequence set forth in SEQ ID NO: 5; optionally with 1, 2, 3, 4, or 5 amino acid additions, substitutions, deletions, mutations, or any combination thereof.

In certain embodiments, the endosomal escape peptide sequence is at least 90%, 95%, 98%, or 99% identical to the amino acid sequence set forth in SEQ ID NO: 5. In certain embodiments, the endosomal escape peptide sequence is at least 90%, 95%, 98%, or 99% identical to the amino acid sequence set forth in SEQ ID NO: 8. In certain embodiments, the endosomal escape peptide sequence is at least 90%, 95%, 98%, or 99% identical to the amino acid sequence set forth in SEQ ID NO: 19. In certain embodiments, the endosomal escape peptide sequence is at least 90%, 95%, 98%, or 99% identical to the amino acid sequence set forth in SEQ ID NO: 21. In certain embodiments, the endosomal escape peptide sequence is at least 90%, 95%, 98%, or 99% identical to the amino acid sequence set forth in SEQ ID NO: 22. In certain embodiments, the endosomal escape peptide sequence is at least 90%, 95%, 98%, or 99% identical to the amino acid sequence set forth in SEQ ID NO: 39. In certain embodiments, the endosomal escape peptide sequence is at least 90%, 95%, 98%, or 99% identical to the amino acid sequence set forth in SEQ ID NO: 43. In certain embodiments, the endosomal escape peptide sequence is at least 90%, 95%, 98%, or 99% identical to the amino acid sequence set forth in SEQ ID NO: 47. In certain embodiments, the endosomal escape peptide sequence is at least 90%, 95%, 98%, or 99% identical to the amino acid sequence set forth in SEQ ID NO: 53.

The novel fusion proteins described herein comprise an endosomal peptide sequence fused to a protein. In certain embodiments, the protein fused to the endosomal escape peptide sequence is a therapeutic protein. In certain embodiments, the protein is an enzyme. In certain embodiments, the protein is a gene-editing protein. In certain embodiments, the protein is selected from the group consisting of Cas9 proteins, nucleases, nickases, methylases, recombinases, deaminases, DNA methyltransferases, histone-modifying enzymes, and transcription factors. In certain embodiments, the protein is a cationic protein. In certain embodiments, a histon-modifying enzyme is selected from the group consisting of histone methyltransferases, histone acetylases, and histone acetyltransferases. In certain embodiments, the protein is a supercharged protein, wherein the supercharged protein has an overall greater net positive charge than its corresponding wild-type protein. In certain embodiments, the overall net positive charge of the supercharged protein is at least +5, +10, +15, +20, +25, +30, +35, or +40. In certain embodiments, the supercharged protein is a fluorescent protein. In certain embodiments, the supercharged protein is a green fluorescent protein (GFP). In certain embodiments, the superpositively charged GFP is +36 GFP. The peptide sequence of +36 GFP is shown below:

(SEQ ID NO: 89)
MGHHHHHHGGASKGERLFRGKVPILVELKGDVNGHKFSVRGKGKGDATRG
KLTLKFICTTGKLPVPWPTLVTTLTYGVQCFSRYPKHMKRHDFFKSAMPK
GYVQERTISFKKDGKYKTRAEVKFEGRTLVNRIKLKGRDFKEKGNILGHK
LRYNFNSHKVYITADKRKNGIKAKFKIRHNVKDGSVQLADHYQQNTPIGR
GPVLLPRNHYLSTRSKLSKDPKEKRDHMVLLEFVTAAGIKHGRDERYK.

In some embodiments, the role of the superpositively charged protein (e.g., +36 GFP) is to promote delivery of the protein or other agent of interest into the cell. For other examples of supercharged proteins contemplated in the present invention, including other examples of superpositively charged green fluorescent proteins, see International Patent Application Nos.: PCT/US2007/070254, PCT/US2009/041984, and PCT/US2010/001250; each of which is incorporated herein by reference.

The fusion proteins of the present invention comprise an endosomal escape peptide sequence fused to a protein, and may further comprise one or more additional agents (i.e., proteins, peptides). In some embodiments, the fusion proteins described herein comprise multiple additional agents per endosomal escape peptide molecule. In some embodiments, the fusion proteins comprise 1, 2, 3, 4, 5, 6, 7, 8, 9, 10, 20, or more additional agents per endosomal escape peptide molecule.

In some embodiments, the fusion proteins of the present invention comprise an endosomal escape peptide sequence fused to a superpositively charged protein that aids in cellular delivery (e.g., a green fluorescent protein, such as +36 GFP), and further comprise one or more additional agents (e.g., peptides, proteins) for delivery to a cell. In some embodiments, the fusion proteins described herein comprise multiple additional agents per endosomal escape peptide/superpositiely charged protein conjugate. In some embodiments, the fusion proteins comprise 1, 2, 3, 4, 5, 6, 7, 8, 9, 10, 20, or more additional agents per endosomal escape peptide/superpositively charged protein conjugate. In certain embodiments, one or more of the additional agents is a therapeutic protein. In certain embodiments, one or more of the additional agents is a gene-editing protein. In certain embodiments, one or more of the additional agent is an enzyme. In certain embodiments, one or more of the enzymes is selected from the group consisting of Cas9 proteins, nucleases, nickases, methylases, recombinases, deaminases, DNA methyltransferases, histone-modifying enzymes (e.g., histone methyltransferases, histone acetylases, histone acetyltransferases), and transcription factors.

As described herein, the fusion proteins of the present invention comprise an endosomal escape peptide fused to a protein, and may further comprise one or more additional agents (i.e., proteins or peptides). In certain embodiments, the fusion protein comprises an endosomal escape peptide, a superpositively charged protein, and a therapeutic protein. In certain embodiments, the fusion protein comprises an endosomal escape peptide, a superpositively charged protein, and an enzyme. In certain embodiments, the fusion protein comprises an endosomal escape peptide sequence, a superpositively charged protein, and a gene-editing protein (e.g., Cas9 proteins, nucleases, nickases, methylases, recombinases, deaminases, DNA methyltransferases, histone-modifying enzymes, and transcription factors). In certain embodiments, the fusion protein comprises an endosomal escape peptide sequence, a superpostively charged green fluorescent protein (GFP), and an additional agent (i.e., peptide or protein). In certain embodiments, the fusion protein comprises an endosomal escape peptide sequence, +36 GFP, and an additional agent (i.e., protein or peptide). In certain embodiments, the fusion protein comprises an endosomal escape peptide sequence, a superpostively charged green fluorescent protein (GFP), and an enzyme. In certain embodiments, the fusion protein comprises an endosomal escape peptide sequence, +36 GFP, and an enzyme. In certain embodiments, the fusion protein comprises an endosomal escape peptide sequence, a superpostively charged green fluorescent protein (GFP), and a gene-editing protein (e.g., Cas9 proteins, nucleases, nickases, methylases, recombinases, deaminases, DNA methyltransferases, histone-modifying enzymes, and transcription factors). In certain embodiments, the fusion protein comprises an endosomal escape peptide sequence, +36 GFP, and a gene-editing protein (e.g., Cas9 proteins, nucleases, nickases, methylases, recombinases, deaminases, DNA methyltransferases, histone-modifying enzymes, and transcription factors.)

The present invention also provides nucleic acids, expression vectors, and cells for expressing any of the fusion proteins described herein. In one aspect, the present invention provides nucleic acids useful in the expression of any of the fusion proteins described herein. In certain embodiments, the nucleic acid used to express any of the proteins described herein is part of an expression vector. In another aspect, the present invention provides vectors (e.g., plasmids, cosmids, viruses, etc.) that comprise any of the inventive sequences described herein. In certain embodiments, the vector includes elements (e.g., promoter, enhancer, ribosomal binding sites, etc.) useful in expressing the proteins described herein in a cell. In another embodiment, the present invention includes cells comprising the inventive sequences or vectors described herein. In certain embodiments, the cells overexpress the inventive sequences described herein. Any cell may be useful in expression the inventive proteins described herein. The cells may be bacterial cells (e.g., E. coli), fungal cells (e.g., P. pastoris), yeast cells (e.g., S. cerevisiae), insect cells, mammalian cells (e.g., CHO cells), or human cells.

### Peptide Conjugates

The present invention also provides novel conjugates comprising a peptide sequence, which promotes endosomal escape (referred to herein as “endosomal escape peptide or “endosomal escape peptide sequence”), fused to an agent (i.e., a small molecule, peptide, or nucleic acid) for delivery to a cell. In certain embodiments, conjugates of the present invention comprise one or more additional agents (e.g., a protein, peptide, small molecule, nucleic acid). One of the additional agents may be a to a superpositively charged protein that aids in cellular delivery (e.g., a green fluorescent protein, such as +36 GFP).

In certain embodiments, conjugates of the present invention comprise an endosomal escape peptide sequence that is at least 90% identical to any one of the amino acid sequences set forth in SEQ ID NOs 1-55 (see Table 1, Table 2, or Table A). In certain embodiments, the endosomal escape peptide sequence is at least 95% identical to any one of the amino acid sequences set forth in SEQ ID NOs: 1-55. In certain embodiments, the endosomal escape peptide sequence is at least 98% identical to any one of the amino acid sequences set forth in SEQ ID NOs: 1-55. In certain embodiments, the endosomal escape peptide sequence is at least 99% identical to any one of the amino acid sequences set forth in SEQ ID NOs: 1-55. In certain embodiments, the endosomal escape peptide sequence is identical to any one of the amino acid sequences set forth in SEQ ID NOs: 1-55. In certain embodiments, the endosomal escape peptide sequence is at least 90% identical to the amino acid sequence set forth in SEQ ID NO: 5. In certain embodiments, the endosomal escape peptide sequence is at least 95% identical to the amino acid sequence set forth in SEQ ID NO: 5. In certain embodiments, the endosomal escape peptide sequence is at least 98% identical to the amino acid sequence set forth in SEQ ID NOs: 5. In certain embodiments, the endosomal escape peptide sequence is at least 99% identical to the amino acid sequence set forth in SEQ ID NOs: 5. In certain embodiments, the endosomal escape peptide sequence is identical to the amino acid sequences set forth in SEQ ID NO: 5. In certain embodiments, the endosomal escape peptide sequence comprises any one of the amino acid sequences set forth in SEQ ID NOs: 1-55; optionally with 1, 2, 3, 4, or 5 amino acid additions, substitutions, deletions, mutations, or any combination thereof. In certain embodiments, the endosomal escape peptide sequence comprises the amino acid sequence set forth in SEQ ID NO: 5; optionally with 1, 2, 3, 4, or 5 amino acid additions, substitutions, deletions, mutations, or any combination thereof.

In certain embodiments, the conjugate comprises an endosomal escape peptide fused to a small molecule (i.e., a therapeutic small molecule or small molecule drug). In certain embodiments, the conjugate comprises an endosomal escape peptide fused to another peptide. In certain embodiments, the conjugate comprises an endosomal escape peptide fused to a nucleic acid (e.g., DNA or RNA, or a hybrib thereof). In certain embodiments, the conjugate comprises an endosomal escape peptide sequence, a small molecule (i.e., a therapeutic small molecule or small molecule drug), and one or more additional agents (e.g., proteins, peptides, small molecules, nucleic acids). In certain embodiments, the conjugate comprises an endosomal escape peptide, a nucleic acid (e.g., DNA or RNA, or a hybrib thereof), and one or more additional agents (e.g., proteins, peptides, small molecules, nucleic acids). In certain embodiments, the conjugate comprises an endosomal escape peptide sequence, a small molecule (i.e., a therapeutic small molecule or small molecule drug), and a protein that aids in cellular delivery (e.g., a superpositively charged protein). In certain embodiments, the conjugate comprises an endosomal escape peptide sequence, a small molecule (i.e., a therapeutic small molecule or small molecule drug), and a cationic protein. In certain embodiments, the conjugate comprises an endosomal escape peptide sequence, a small molecule (i.e., a therapeutic small molecule or small molecule drug), and a superpositively charged protein. In certain embodiments, the conjugate comprises an endosomal escape peptide sequence, a small molecule (i.e., a therapeutic small molecule or small molecule drug), and a superpositively charged green fluorescent protein (GFP) (e.g., +36 GFP).

In certain embodiments, the conjugate comprises an endosomal escape peptide sequence, a nucleic acid (e.g., DNA or RNA, or a hybrib thereof), and a protein that aids in cellular delivery (e.g., a superpositively charged protein). In certain embodiments, the conjugate comprises an endosomal escape peptide sequence, a nucleic acid (e.g., DNA or RNA, or a hybrib thereof), and a cationic protein. In certain embodiments, the conjugate comprises an endosomal escape peptide sequence, a nucleic acid (e.g., DNA or RNA, or a hybrib thereof), and a superpositively charged protein. In certain embodiments, the conjugate comprises an endosomal escape peptide sequence, a nucleic acid (e.g., DNA or RNA, or a hybrib thereof), and a superpositively charged green fluorescent protein (GFP) (e.g., +36 GFP).

### Methods for Preparing Fusion Proteins and Peptide Conjugates

In one aspect, the present invention provides methods for preparing fusion proteins and conjugates described herein. In general, methods for preparing fusion proteins and conjugates described herein involve conjugating an endosomal escape peptide to a protein or other agent of interest. One of skill in the art will appreciate that proteins and other agents of interest can be fused to endosomal escape peptides via any method for conjugation or ligation known in the art. Any covalent or non-covalent bond-forming reaction is contemplated as being within the scope of the present invention, including, but not limited to, nucleophilic displacement reactions, addition reactions, metathesis reactions, cycloadditon reactions, and coupling reactions. In certain embodiments, the protein or other agent of interest is conjugated to the endosomal escape peptide via a peptide/amide bond forming reaction. In other embodiments, the protein or other agent of interest is conjugated to the endosomal escape peptide via a click chemistry reaction, wherein “click chemisty reaction” is as defined herein. Other bioconjugation techniques can be employed to fuse the endosomal escape peptide to agents of interest; see, e.g., Stephanopoulos et al. Nature Chemical Biology 2011, 7(12): 876-884.

In certain embodiments, the methods for preparing the fusion proteins and conjugates described herein involve sortase-mediate transpeptidation. A typical method for preparing a fusion protein described herein using sortase-mediated transpeptidation comprises contacting:
(1) a peptide of the structure: [first peptide]-[first sortase recognition motif]; with
(2) a substrate of the structure: [second sortase recognition motif]-[second agent], wherein the second agent comprises one or more agents selected from the group consisting of proteins, peptides, nucleic acids, and small molecules; and
(3) a sortase;
   under conditions suitable for the sortase to catalyze a transpeptidation reaction, wherein “sortase” and “sortase recognition motif” are as defined herein.

For exemplary sortases, sortase recognition motifs, reagents, and conditions for sortase-mediated transpeptidation which may be employed in the methods of the present invention, see, e.g., Ploegh et al., International PCT Patent Application, PCT/US2010/000274, filed Feb. 1, 2010, published as WO 2010/087994, on Aug. 5, 2010; Ploegh et al., International Patent Application PCT/US2011/033303, filed Apr. 20, 2011, published as WO 2011/133704, on Oct. 27, 2011; Liu et al., U.S. provisional patent application, U.S. Ser. No. 61/662,606, filed on Jun. 21, 2012; and Liu et al., U.S. provisional patent application, U.S. Ser. No. 61/880,515, filed on Sep. 20, 2013; and Liu, et al. International Patent Application No. PCT/US2013/067461; each of which is incorporated herein by reference.

As generally defined herein, the “first peptide” is any one of the endosomal escape peptide sequences described herein. In certain embodiments, the first peptide comprises a peptide sequence that is at least 90% identical to any one of the amino acid sequences set forth in SEQ ID NOs: 1-55 (see Table 1 and Table 2). In certain embodiments, the first peptide is at least 95% identical to any one of the amino acid sequences set forth in SEQ ID NOs: 1-55. In certain embodiments, the first peptide sequence is at least 98% identical to any one of the amino acid sequences set forth in SEQ ID NOs: 1-55. In certain embodiments, the first peptide sequence is at least 99% identical to any one of the amino acid sequences set forth in SEQ ID NOs: 1-55. In certain embodiments, the first peptide sequence is identical to any one of the amino acid sequences set forth in SEQ ID NOs: 1-55. In certain embodiments, the first peptide sequence is at least 90% identical to the amino acid sequence set forth in SEQ ID NO: 5. In certain embodiments, the first peptide sequence is at least 95% identical to the amino acid sequence set forth in SEQ ID NO: 5. In certain embodiments, the first peptide sequence is at least 98% identical to the amino acid sequence set forth in SEQ ID NOs: 5. In certain embodiments, the first peptide sequence is at least 99% identical to the amino acid sequence set forth in SEQ ID NOs: 5. In certain embodiments, the first peptide sequence is identical to the amino acid sequences set forth in SEQ ID NO: 5. In certain embodiments, the first peptide sequence comprises any one of the amino acid sequences set forth in SEQ ID NOs: 1-55; optionally with 1, 2, 3, 4, or 5 amino acid additions, substitutions, mutations, or any combination thereof. In certain embodiments, the first peptide sequence comprises the amino acid sequence set forth in SEQ ID NO: 5; optionally with 1, 2, 3, 4, or 5 amino acid additions, substitutions, mutations, or any combination thereof.

As generally defined herein, the “first sortase recognition motif” is any amino acid sequence known in the art which can be used as a C-terminal or N-terminal handle for sortase-catalyzed transpeptidation. In certain embodiments of the present invention, the first sortase recognition motif comprises an LPXT motif, wherein X is any amino acid. In certain embodiments, the first sortase recognition motif comprises the sequence: LPETG (SEQ ID NO: 90). In certain embodiments, the first sortase recognition motif is of the amino acid sequence: LPETGG (SEQ ID NO: 91). In other embodiments, the first sortase recognition motif comprises an LPXS motif, wherein X is any amino acid. In certain embodiments, the first sortase recognition motif comprises one of the following amino acid sequences: LPESG (SEQ ID NO: 95), LAETG (SEQ ID NO: 96), or LAESG (SEQ ID NO: 97),In certain embodiments, the first sortase regconition motif is an N-terminus sortase recognition motif (e.g., a polyglycine, such as GGG).

In some instances, the first peptie (i.e., endosomal escape peptide) is conjugated to the first sortase recognition motif, resulting in a peptide of structure: [first peptide]-[first sortase recognition motif], which is then ligated to the protein or other agent of interest via sortase-mediated transpeptidation. Any method known in the art for conjugation or ligation can be used to conjugate the first peptide to the first sortase recognition motif, including common covalent bond-forming reactions (e.g., nucleophilic displacement reactions, addition reactions, metathesis reactions, cycloadditon reactions, coupling reactions). In certain embodiments, the reaction is an amide/peptide bond forming reaction. In certain embodiments, the reaction is a click chemistry reaction. Other bioconjugation techniques may be employed to fuse the endosomal escape peptide to sortase recognition motifs; see, e.g., Stephanopoulos et al. Nature Chemical Biology 2011, 7(12): 876-884.

In some instances, the second agent is conjugated to the second sortase recognition motif, resulting in a peptide of structure: [second sortase recognition motif]-[second agent], which is then ligated to the endosomal escape peptide via sortase-mediated transpeptidation. Any method known in the art for conjugation or ligation can be used to conjugate the second agent to the second sortase recognition motif. These methods include, but are not limited to, common covalent bond-forming reactions (e.g., nucleophilic displacement reactions, addition reactions, metathesis reactions, cycloadditon reactions, coupling reactions). In certain embodiments, the reaction used to conjugate the second agent to a sortase recognition motif is an amide/peptide bond forming reaction or a click chemistry reaction; however, other bioconjugation techniques may be employed; see, e.g., Stephanopoulos et al. Nature Chemical Biology 2011, 7(12): 876-884.

As generally defined herein, the “second sortase recognition motif” is any amino acid sequence known in the art which may be used as C-terminal or N-terminal handle for sortase-catalyzed transpeptidation. In certain embodiments, the second sortase recognition motif comprises a polyglycine sequence, wherein the polyglycine sequence comprises two or more consecutive glycine residues. In certain embodiments, the second sortase recognition motif comprises 2, 3, 4, 5, 6, 7, 8, 9, or 10 consecutive glycine residues, inclusive. In certain embodiments, the second sortase recognition motif comprises three consecutive glycine residues. In certain embodiments, the second sortase recognition motif is of the amino acid sequence: GGG (SEQ ID NO: 92). In certain embodiments, the second sortase recognition motif is an N-terminal motif (e.g., an LPXT motif, such as LPETG (SEQ ID NO: 90) or LPETGG (SEQ ID NO: 91); or an LPXS motif, such as LPESG (SEQ ID NO: 95), LAETG (SEQ ID NO: 96), or LAESG (SEQ ID NO: 97)).

As described herein, the fusion proteins and conjugates of the present invention can be prepared by contacting a peptide of the structure: [first peptide]-[first sortase recognition motif]; with a substrate of the structure: [second sortase recognition motif]-[second agent]; and a sortase; under conditions suitable for the sortase to catalyze a transpeptidation reaction. In certain embodiments, the sortase is sortase A. In certain embodiments, the sortase is an evolved sortase A enzyme (eSrtA) described in Chen et al. Proceedings of the National Academy of Sciences 2011, 108, 11399, incorporated herein by reference. For other exemplary sortases, see, e.g., Liu et al., U.S. provisional Patent Application 61/662,606, filed on Jun. 21, 2012; and Liu et al., U.S. provisional Patent Application 61/880,515, filed on Sep. 20, 2013; and Liu, et al. International Patent Application No. PCT/US2013/067461; the entire contents of each of which are incorporated herein by reference.

In other embodiments of the invention, the first peptide is attached to the second sortase recognition motif, and the second agent is attached to the first sortase recognition motif.

### Preparation of Fusion Proteins

When the “second agent” is a protein, a fusion protein is formed in the sortase-mediated ligation described herein. In certain embodiments, the second agent is a therapeutic protein. In certain embodiments, the second agent is a gene-editing protein (e.g., Cas9 proteins, nucleases, nickases, methylases, recombinases, deaminases, DNA methyltransferases, histone-modifying enzymes, transcription factors). In certain embodiments, the second agent is a protein that aids in cellular delivery (e.g., a superpositively charged protein, such as superpositively charged GFP).

In certain embodiments, the second sortase recognition motif is a polyglycine sequence comprising two or more consecutive glycine residues, and the second agent is a protein. In certain embodiments, the second sortase recognition motif is a polyglycine sequence comprising two or more consecutive glycine residues, and the second agent comprises a protein that aids in cellular delivery (e.g., a superpositively charged GFP). In certain embodiments, the second sortase recognition motif is a polyglycine sequence comprising two or more consecutive glycine residues, and the second agent comprises a superpositively charged protein. In certain embodiments, the second sortase recognition motif is a polyglycine sequence comprising two or more consecutive glycine residues, and the second agent comprises a superpositively charged green fluorescent protein (GFP). In certain embodiments, the second sortase recognition motif is GGG (SEQ ID NO: 92), and the second agent is a superpositively charged green fluorescent protein (GFP). In certain embodiments, the second sortase recognition motif is GGG (SEQ ID NO: 92), and the second agent is +36 GFP.

In certain embodiments, the second agent further comprises one or more additional agents (i.e., proteins, peptides). In certain embodiments, the second agent further comprises one or more therapeutic proteins. In certain embodiments, the second agent further comprises one or more gene-editing proteins (e.g., Cas9 proteins, nucleases, nickases, methylases, recombinases, deaminases, DNA methyltransferases, histone-modifying enzymes, and transcription factors.) In certain embodiments, the second agent further comprises one or more proteins that aid in cellular delivery (e.g., a superpositively charged protein, such as superpositively charged GFP).

### Preparation of Conjugates

In addition to conjugating proteins to endosomal peptides to form fusion proteins, other agents of interest (i.e., small molecules and nucleic acids) can be conjugated to endosomal escape peptides to form conjugates of the present invention. Therefore, in some embodiments, the “second agent” is a small molecule (i.e., a therapeutic small molecule or small molecule drug). In other embodiments, the second agent is a nucleic acid (e.g., DNA, RNA, or a hybrid thereof).

In some embodiments, the second agent comprises a small molecule and one or more additional agents selected from the group consisting of proteins, peptides, small molecules, and nucleic acids. In other embodiments, the second agent comprises a nucleic acid and one or more additional agent selected from the group consisting of proteins, peptides, small molecules, and nucletic acids. In certain embodiments, the additional agent is a protein that aids in cellular delivery (e.g., a superpositively charged protein, such as superpositively charged GFP). In certain embodiments, the second agent comprises a small molecule fused to a protein at aids in cellular delivery (e.g., a superpositively charged protein, such as superpositively charged GFP). In certain embodiments, the second agent comprises a nucleic acid fused to a protein at aids in cellular delivery (e.g., a superpositively charged protein, such as superpositively charged GFP).

### Novel Peptides

The present invention provides novel peptides of structure: [first peptide]-[first sortase recognition motif], which are useful in preparing the fusion proteins described herein. In certain embodiments of the invention, the “first peptide” is any one of the endosomal escape peptide sequences described herein. In certain embodiments, the first peptide comprises a peptide sequence that is at least 90% identical to any one of the amino acid sequences set forth in SEQ ID NOs 1-55 (see Table 1 and Table 2). In certain embodiments, the peptide sequence is at least 95% identical to any one of the amino acid sequences set forth in SEQ ID NOs: 1-55. In certain embodiments, the peptide sequence is at least 98% identical to any one of the amino acid sequences set forth in SEQ ID NOs: 1-55. In certain embodiments, the peptide sequence is at least 99% identical to any one of the amino acid sequences set forth in SEQ ID NOs: 1-55. In certain embodiments, the peptide sequence is identical to any one of the amino acid sequences set forth in SEQ ID NOs: 1-55. In certain embodiments, the peptide sequence is at least 90% identical to the amino acid sequence set forth in SEQ ID NO: 5. In certain embodiments, the peptide sequence is at least 95% identical to the amino acid sequence set forth in SEQ ID NO: 5. In certain embodiments, the peptide sequence is at least 98% identical to the amino acid sequence set forth in SEQ ID NOs: 5. In certain embodiments, the peptide sequence is at least 99% identical to the amino acid sequence set forth in SEQ ID NOs: 5. In certain embodiments, the peptide sequence is identical to the amino acid sequences set forth in SEQ ID NO: 5. In certain embodiments, the peptide sequence comprises any one of the amino acid sequences set forth in SEQ ID NOs: 1-55; optionally with 1, 2, 3, 4, or 5 amino acid additions, substitutions, mutations, or any combination thereof. In certain embodiments, the peptide sequence comprises the amino acid sequence set forth in SEQ ID NO: 5; optionally with 1, 2, 3, 4, or 5 amino acid additions, substitutions, mutations, or any combination thereof.

As generally defined herein, the “first sortase recognition motif” is any amino acid sequence known in the art which may be used as a C-terminal or N-terminal handle for sortase-catalyzed transpeptidation. In certain embodiments of the present invention, the first sortase recognition motif comprises an LPXT motif, wherein X is any amino acid. In certain embodiments, the first sortase recognition motif comprises the sequence: LPETG (SEQ ID NO: 90). In certain embodiments, the first sortase recognition motif is of the amino acid sequence: LPETGG (SEQ ID NO: 91). In other embodiments, the first sortase recognition motif comprises an LPXS motif, wherein X is any amino acid. In certain embodiments, the first sortase recognition motif comprises one of the following amino acid sequences: LPESG (SEQ ID NO: 95), LAETG (SEQ ID NO: 96), or LAESG (SEQ ID NO: 97).

In certain embodiments, the peptide of structure: [first peptide]-[first sortase recognition motif] is at least 90%, 95%, 98%, or 99% identical to the peptide sequence: GLFDIIKKIAESFLPETGG (SEQ ID NO: 93). In certain embodiments, the peptide of structure [first peptide]-[first sortase recognition motif] is identical to the peptide sequence set forth in SEQ ID NO: 93.

The present invention also provides novel peptides that promote endosomal escape of proteins and other agents. In some embodiments, these novel peptides comprise peptide sequences that are at least 90% identical to any one of the amino acid sequences set forth in SEQ ID NOs: 37-55. The peptide of claim 62, wherein the peptide sequence is at least 95% identical to any one of the amino acid sequences set forth in SEQ ID NOs: 37-55. In certain embodiments, the peptide sequence is at least 98% identical to any one of the amino acid sequences set forth in SEQ ID NOs: 37-55. In certain embodiments, the peptide sequence is at least 98% identical to any one of the amino acid sequences set forth in SEQ ID NOs: 37-55. In certain embodiments, the peptide sequence is at least 99% identical to any one of the amino acid sequences set forth in SEQ ID NOs: 37-55. In certain embodiments, the peptide sequence is identical to any one of the amino acid sequences set forth in SEQ ID NOs: 37-55.

### Applications

The present invention provides proteins and and conjugates comprising endosomal escape peptide sequences that enhance endosomal escape of a protein or other agent, as well as methods for using such fusion proteins and conjugates. The inventive proteins and conjugates may be used to treat or prevent any disease that can benefit from the delivery of a therapeutic agent (e.g., protein, peptide, nucleic acid, small molecule) into the cytosol of a cell. Fusion proteins and conjugates of the present invention may comprise gene-editing proteins (e.g., Cas9 proteins, nucleases, nickases, methylases, recombinases, deaminases, DNA methyltransferases, histone-modifying enzymes, transcription factors), and therefore the fusion proteins and conjugates may also be used to reprogram cells or edit the genome of a cell. The inventive fusion proteins and conjugates may be used to transfect cells for research purposes.

In some embodiments, fusion proteins and conjugates in accordance with the invention may be used for research purposes, e.g., to efficiently deliver proteins and other agents of interest to cells in a research context. In some embodiments, proteins and conjugates in accordance with the present invention may be used for therapeutic purposes. In certain embodiments, the proteins and conjugates of the present invention may be administered to a subject. In certain embodiments, the administering is performed under conditions sufficient for the protein to penetrate a cell of the subject. In some embodiments, proteins and conjugates in accordance with the present invention may be used for treatment of any of a variety of diseases, disorders, and/or conditions, including, but not limited to, one or more of the following: autoimmune disorders (e.g. diabetes, lupus, multiple sclerosis, psoriasis, rheumatoid arthritis); inflammatory disorders (e.g. arthritis, pelvic inflammatory disease); infectious diseases (e.g. viral, bacterial, and fungal infections; sepsis); neurological disorders (e.g. Alzheimer's disease, autism); cardiovascular disorders (e.g. atherosclerosis, thrombosis, clotting disorders, angiogenic disorders such as macular degeneration); proliferative disorders (e.g. cancer); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); and blood and lymphatic disorders (e.g. anemia, hemophilia).

In some embodiments, the protein or conjugate of the present invention is detectable. For example, the protein or conjugate may comprise at least one fluorescent moiety. In some embodiments, the fusion protein or conjugate comprises a supercharged protein which has inherent fluorescent qualities (e.g., GFP). In some embodiments, the fusion protein is associated with at least one fluorescent moiety (e.g., conjugated to a fluorophore). In some embodiments, the fusion protein or conjugate is associated with at least one chromophore, phosphorescent moiety, dye, or other detectable moiety. Alternatively or additionally, the fusion protein or conjugate may comprise at least one radioactive moiety (e.g., protein may comprise ³⁵S; nucleic acid may comprise ³²P). Such detectable moieties may be useful for detecting and/or monitoring delivery of the fusion protein or conjugate to a target site (e.g., a target cite within the cell).

### Pharmaceutical Compositions, Administration, and Kits

The present invention provides fusion proteins and conjugates with enhanced capabilities for endosomal escape and cytosolic delivery. Thus, the present invention provides pharmaceutical compositions comprising a fusion proteins or conjugates as described herein, and one or more pharmaceutically acceptable excipients. Pharmaceutical compositions may optionally comprise one or more additional therapeutically active substances. In some embodiments, compositions are administered to humans.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween®20], polyoxyethylene sorbitan [Tween®60], polyoxyethylene sorbitan monooleate [Tween®80], sorbitan monopalmitate [Span®40], sorbitan monostearate [Span®60], sorbitan tristearate [Span®65], glyceryl monooleate, sorbitan monooleate [Span®80]), polyoxyethylene esters (e.g. polyoxyethylene monostearate [Myrj®45], polyoxyethylene hydrogenated castor oil, polyethoxylated castor oil, polyoxymethylene stearate, and Solutol®), sucrose fatty acid esters, polyethylene glycol fatty acid esters (e.g. Cremophor®), polyoxyethylene ethers, (e.g. polyoxyethylene lauryl ether [Brij®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F 68, Poloxamer® 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall® 115, Germaben® II, Neolone™, Kathon™, and/or Euxyl®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

The present invention provides methods comprising administering proteins in accordance with the invention to a subject in need thereof. Proteins or pharmaceutical compositions thereof may be administered to a subject using any amount and any route of administration effective for treating a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Fusion proteins and/or pharmaceutical compositions thereof in accordance with the present invention may be administered by any route. In some embodiments, complexes and/or pharmaceutical compositions thereof are administered by one or more of a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments, complexes and/or pharmaceutical compositions thereof are administered by systemic intravenous injection. In specific embodiments, complexes and/or pharmaceutical compositions thereof may be administered intravenously and/or orally. In specific embodiments, complexes and/or pharmaceutical compositions thereof may be administered in a way which allows the complex to cross the blood-brain barrier. However, the invention encompasses the delivery of complexes and/or pharmaceutical compositions thereof by any appropriate route taking into consideration likely advances in the sciences of drug delivery.

In certain embodiments, compositions in accordance with the invention may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Fusion proteins and conjugates of the present invention may be administered in combination with one or more other therapeutic agents. By “in combination with,” it is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In general, each agent will be administered at a dose and/or on a time schedule determined for that agent. In some embodiments, the invention encompasses the delivery of pharmaceutical compositions in combination with agents that may improve their bioavailability, reduce and/or modify their metabolism, inhibit their excretion, and/or modify their distribution within the body.

In will further be appreciated that therapeutically active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer in accordance with the invention may be administered concurrently with a chemotherapeutic agent), or they may achieve different effects (e.g., control of any adverse effects).

The invention provides kits for conveniently and/or effectively carrying out methods of the present invention. Typically kits will comprise sufficient amounts and/or numbers of components to allow a user to perform multiple treatments of a subject(s).

In some embodiments, kits comprise one or more of (i) a protein of the present invention, as described herein; (ii) at least one pharmaceutically acceptable excipient; (iii) a syringe, needle, applicator, etc. for administration of a pharmaceutical composition to a subject; and (iv) instructions for preparing pharmaceutical composition and for administration of the composition to the subject.

In some embodiments, kits comprise one or more of (i) a pharmaceutical composition comprising a fusion protein or conjugate described herein; (ii) a syringe, needle, applicator, etc. for administration of the pharmaceutical composition to a subject; and (iii) instructions for administration of the pharmaceutical composition to the subject.

In some embodiments, kits include a number of unit dosages of a pharmaceutical composition comprising a protein of the present invention. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

Kits may comprise one or more vessels or containers so that certain of the individual components or reagents may be separately housed. Kits may comprise a means for enclosing individual containers in relatively close confinement for commercial sale (e.g., a plastic box in which instructions, packaging materials such as styrofoam, etc., may be enclosed). Kit contents are typically packaged for convenience use in a laboratory.

### EXAMPLES

In order that the invention described herein may be more fully understood, the following examples are set forth. The synthetic examples described in this application are offered to illustrate the compounds and methods provided herein and are not to be construed in any way as limiting their scope.

Discovery and Characterization of Peptides that Enhance Endosomal Escape

Antimicrobial peptides (AMPs) are a class of membrane-active peptides that penetrate microbial membranes to provide defense against bacteria, fungi, and viruses, often with high selectivity. See, e.g., Zasloff, M. Nature 2002, 415, 389. Given that many AMPs exhibit minimal toxicity to mammalian cells, it is possible that the altered endosomal environment or endosomal membrane curvature could induce some AMPs to be endosomolytic without exhibiting significant mammalian cell toxicity at useful concentrations. See, e.g., Lohner et al. Combinatorial chemistry & high throughput screening 2005, 8, 241. A screen of AMPs for their ability to increase protein delivery into the cytosol was performed

A major challenge to developing agents that enhance endosomal escape is the lack of well-established assays that can distinguish proteins trapped in the endosomes from proteins released into the cytosol. Commonly used enzyme delivery assays involve substrates and products that can freely diffuse through membranes and cannot differentiate between endosomal and cytosolic proteins. To overcome this challenge, multiple independent assays that reflect the interaction of a variety of cargo with a variety of cytosolic targets were used to evaluate endosomal escape of AMP-protein fusions.

Aurein 1.2 (GLFDIIKKIAESF (SEQ ID NO: 5)) and derivatives thereof were discovered as peptides that enhance the endosomal escape of a variety of cargo fused to +36 GFP. The structure-function relationships within aurein 1.2 was elucidated using alanine scanning and mutational analysis. Results from three independent delivery assays confirmed that treatment of mammalian cells with cargo proteins fused to aurein 1.2-+36 GFP result in more efficient cytosolic delivery than the same proteins fused to +36 GFP alone. Finally, the ability of aurein 1.2 to enhance non-endosomal protein delivery was explored in vivo. Cre recombinase enzyme was delivered into hair cells in the cochlea (inner ear) of live mice with much greater (>20-fold) potency when fused with aurein 1.2 than in the absence of the peptide. These results together provide a simple molecular strategy for enhancing the cytosolic delivery of proteins in cell culture and in vivo that is genetically encoded, localized to cargo molecules, and does not require systemic treatment with cytotoxic small molecules.

### Preparation of Antimicrobial Peptide Conjugates of Supercharged GFP-Cre Fusion Proteins

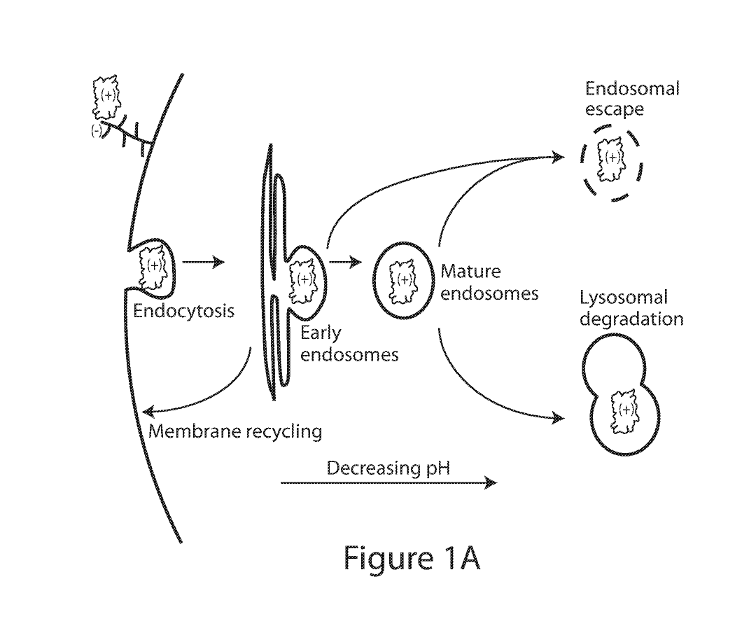

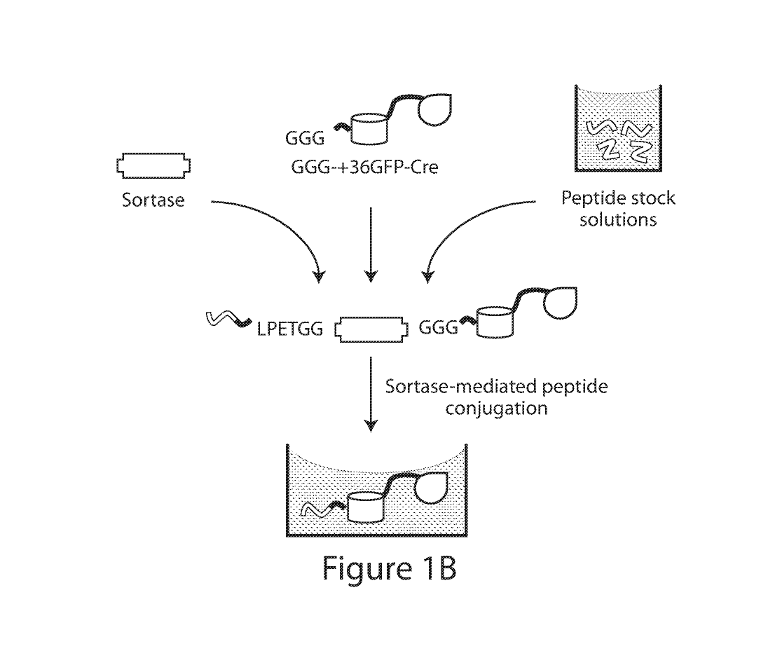

AMPs from the Antimicrobial Peptide Database that are ≤25 amino acids long, lack post-translational modifications, and are not known to be toxic to mammalian cells were sought. Based on these criteria, 36 AMPs ranging from 9 to 25 amino acids in length were identified (Table 1). See, e.g., Wang et al. Nucleic acids research 2004, 32, D590. Each of the peptides was synthesized on solid phase with an LPETGG (SEQ ID NO: 91) sequence appended to their C-terminus to enable sortase-catalyzed conjugation (FIG. 1B). See, e.g., Chen et al. Proceedings of the National Academy of Sciences 2011, 108, 11399. Assembly of proteins using sortase proved more amenable to rapid screening than the construction and expression of the corresponding fusions, especially since several AMP fusions do not express efficiently in E. coli.

TABLE 1
List of peptides chosen from the Antimicrobial
Peptide Database (APD)

| | | SEQ | | |
|---|---|---|---|---|
| | APD | ID | | Conjugation |
| Label | number | NO: | Sequence | efficiency |
| A | AP00408 | 1 | FLFPLITSFLSKVL | 55% |
| B | AP00405- | 2 | FISAIASMLGKFL | 70% |
| | 11 | | | |
| C | AP00327 | 3 | GWFDVVKHIASAV | — |
| D | AP01434 | 4 | FFGSVLKLIPKIL | — |
| E | AP00013 | 5 | GLFDIIKKIAESF | 77% |
| F | AP00025 | 6 | HGVSGHGQHGVHG | 20% |
| G | AP00094 | 7 | FLPLIGRVLSGIL | — |
| H | AP00012 | 8 | GLFDIIKKIAESI | 28% |
| I | AP00014 | 9 | GLLDIVKKVVGAFGSL | — |
| J | AP00015 | 10 | GLFDIVKKVVGALGSL | 13% |
| K | AP00016 | 11 | GLFDIVKKVVGAIGSL | — |
| L | AP00017 | 12 | GLFDIVKKVVGTLAGL | 18% |
| M | AP00018 | 13 | GLFDIVKKVVGAFGSL | — |
| N | AP00019 | 14 | GLFDIAKKVIGVIGSL | — |
| O | AP00020 | 15 | GLFDIVKKIAGHIAGSI | — |
| P | AP00021 | 16 | GLFDIVKKIAGHIASSI | — |
| Q | AP00022 | 17 | GLFDIVKKIAGHIVSSI | — |
| R | AP00101 | 18 | FVQWFSKFLGRIL | 51% |
| S | AP00351 | 19 | GLFDVIKKVASVIGGL | 11% |
| T | AP00352 | 20 | GLFDIIKKVASVVGGL | — |
| U | AP00353 | 21 | GLFDIIKKVASVIGGL | 4% |
| V | AP00567 | 22 | VWPLGLVICKALKIC | 4% |
| W | AP00597 | 23 | NFLGTLVNLAKKIL | 34% |
| X | AP00818 | 24 | FLPLIGKILGTIL | 14% |
| Y | AP00866 | 25 | FLPIIAKVLSGLL | 86% |
| Z | AP00870 | 26 | FLPIVGKLLSGLL | — |
| AA | AP00875 | 27 | FLSSIGKILGNLL | 88% |
| AB | AP00898 | 28 | FLSGIVGMLGKLF | 70% |
| AC | AP01211 | 29 | TPFKLSLHL | 81% |
| AD | AP01249 | 30 | GILDAIKAIAKAAG | 20% |
| AE | AP00013- | 31 | LFDIIKKIAESF | 63% |
| | G | | | |
| AF | AP00013- | 32 | LFDIIKKIAESGFLFDIIK | — |
| | 2x | | KIAES | |
| AG | AP00722- | 33 | GLLNGLALRLGKRALKKII | — |
| | 75 | | KR | |
| AH | His13 | 34 | GHHHHHHHHHHHHH | — |
| AI | AP00512 | 35 | FKCRRWQWRM | 42% |
| AJ | AP00553 | 36 | KTCENLADTY | — |

Peptides were synthesized with a C-terminal LPETGG tag to enable conjugation with an evolved sortase (eSrtA). Conjugation efficiencies were calculated based on LC-MS results using peak abundance as determined through MaxEnt protein deconvolution.

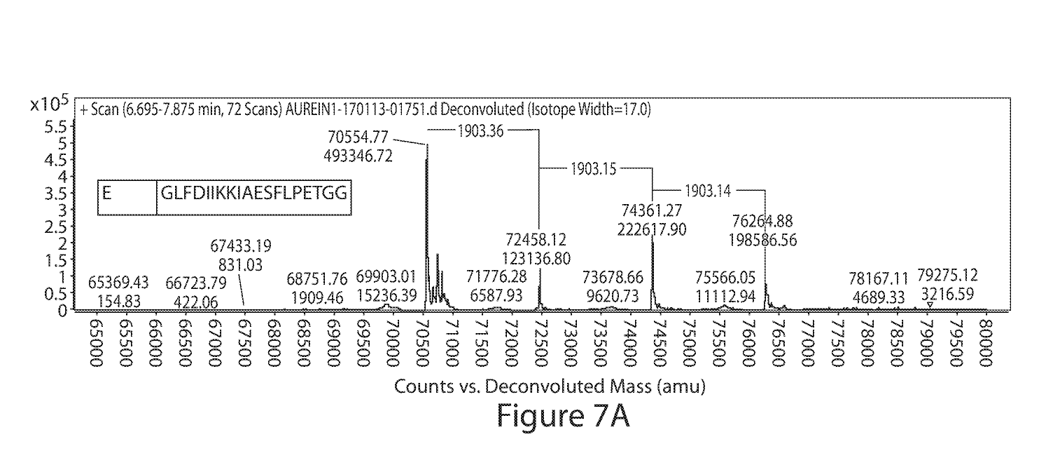

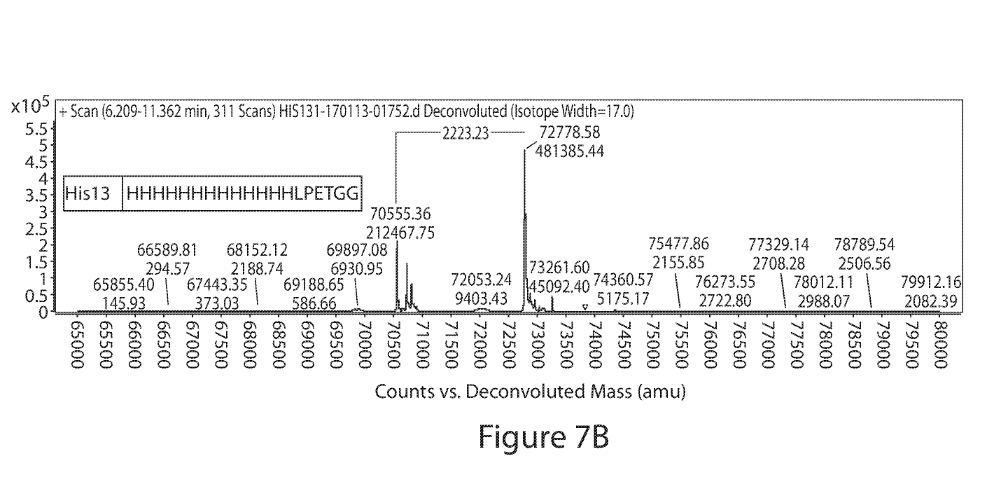

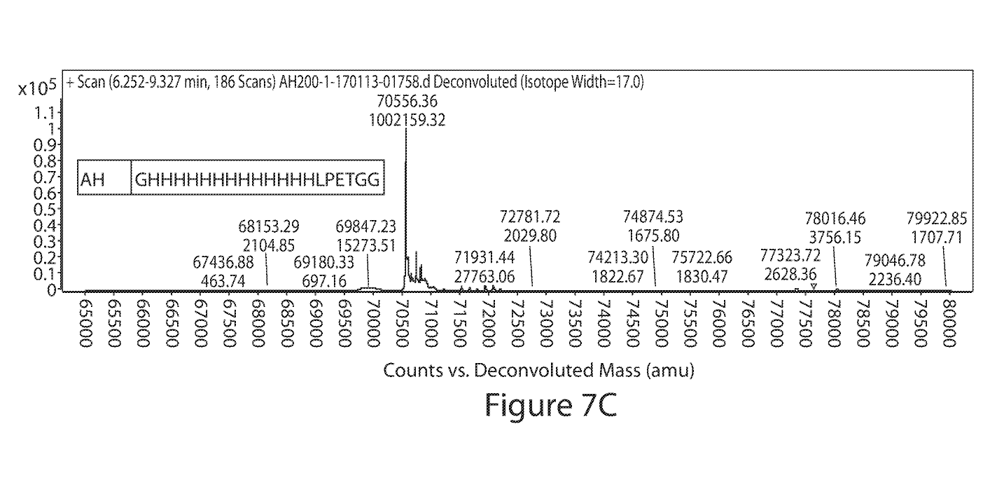

The peptides were conjugated to purified GGG-(+36 GFP)-Cre using an evolved sortase A enzyme (eSrtA). See, e.g., Chen et al., Proceedings of the National Academy of Sciences 2011, 108, 11399. Sortase catalyzes the transpeptidation between a substrate containing the C-terminal LPETGG (SEQ ID NO: 91) and a substrate containing an N-terminal glycine to form a native peptide bond linkage and a protein identical to the product of translational fusion. The efficiency of eSrtA-mediated conjugation varied widely among the peptides (FIG. 7). Of the 36 peptides chosen for screening, 20 showed detectable (4% to 88%) sortase-mediated conjugation to +36 GFP-Cre, as observed by LC-MS, to generate desired peptide-LPETGGG (SEQ ID NO: 98)-(+36 GFP)-Cre fusion proteins (Table 1). Unreacted peptide was removed by ultrafiltration with a 30-kD molecular weight cut off membrane.

### Primary Screen for Endosomal Escape

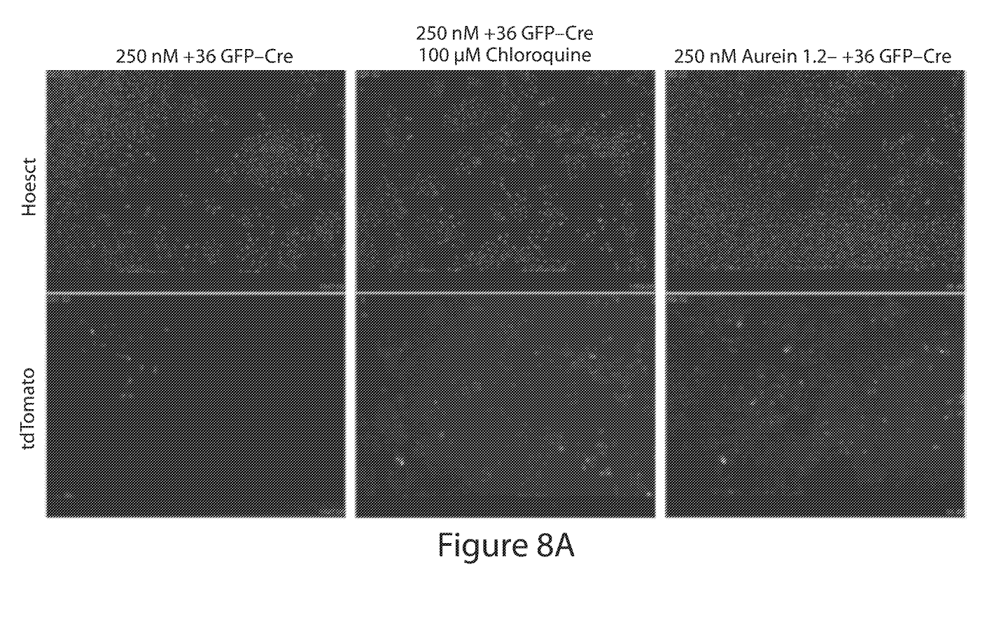

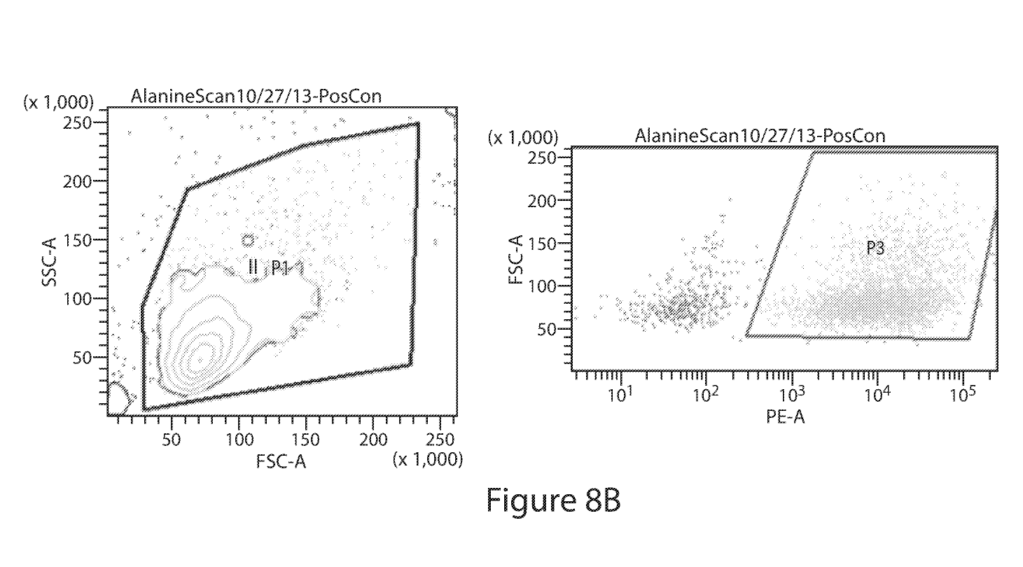

The ability of each peptide-(+36 GFP)-Cre recombinase fusion when added to culture media to effect recombination was assayed in BSR.LNL.tdTomato cells, a hamster kidney cell line derived from BHK-21 (FIG. 8). Because Cre recombinase must enter the cell, escape endosomes, enter the nucleus, and catalyze recombination to generate tdTomato fluorescence, this assay reflects the availability of active, non-endosomal recombinase enzyme that reaches the nucleus. As a positive control, we treated cells with +36 GFP-Cre and chloroquine, a known endosome-disrupting small molecule. See, e.g., Dijkstra at al. Biochimica et Biophysica Acta (BBA)-Molecular Cell Research 1984, 804, 58.

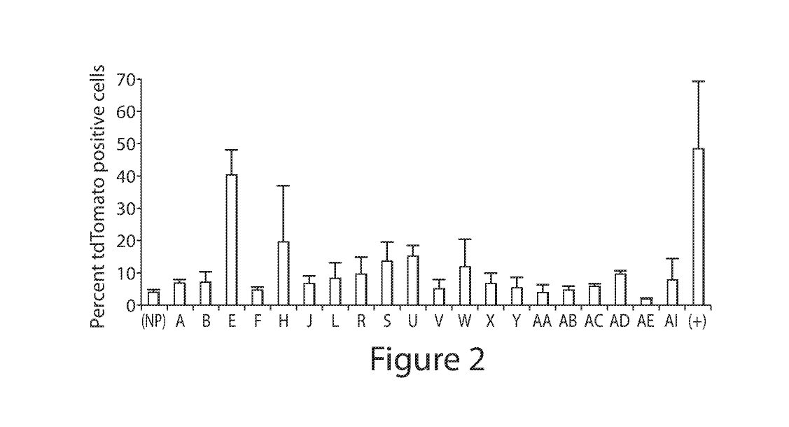

The reporter BSR.LNL.tdTomato cells were incubated with 250 nM of each peptide-(+36 GFP)-Cre protein in serum-free media. In the absence of any conjugated peptide, treatment of reporter cells with 250 nM+36 GFP-Cre protein resulted in 4.5% of the cells expressing tdTomato, consistent with previous reports. The same concentration of protein incubated with 100 μM chloroquine as a positive control resulted in an average of 48% recombined cells (FIG. 2). The results of chloroquine treatment varied substantially between independent replicates. As chloroquine is known to be toxic to cells above 100 μM, it is possible that this variability arises from the small differences between chloroquine's efficacious and toxic dosages.

Ten of the screened peptide conjugates resulted in recombination efficiencies that were significantly above that of +36 GFP-Cre (FIG. 2). The most potent functional delivery of Cre was observed with aurein 1.2-+36 GFP-Cre (Table 1, entry E). Treatment with aurein 1.2-+36 GFP-Cre resulted in an average of 40% recombined cells, comparable to that of the chloroquine positive control (FIG. 2). To investigate the impact of differential conjugation efficiency on peptide performance, we compared citropin 1.3 (Table 1, entry U), which displayed a moderate level of recombination and the lowest level of conjugation (4%), to aurein 1.2, which has the highest level of recombination and also a high level of conjugation (77%).

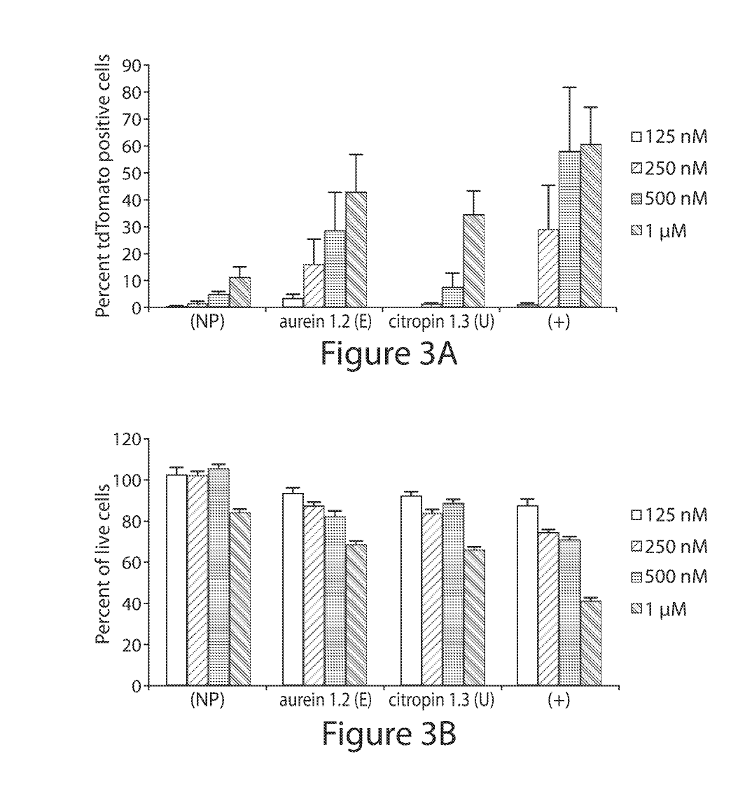

Both aurein 1.2-+36 GFP-Cre and citropin 1.3-+36 GFP-Cre were cloned, expressed, and purified as fusion proteins. The recombination signal from treatment with 250 nM of expressed aurein 1.2-+36 GFP-Cre was 10.4-fold above that of +36 GFP-Cre. In contrast, treatment with 250 nM expressed citropin 1.3-+36 GFP-Cre did not induce any enhanced Cre delivery. When the treatment concentration was increased to 1 μM, aurein 1.2-+36 GFP-Cre and citropin 1.3-+36 GFP-Cre resulted in 3.8-fold and 3.0-fold higher recombination levels, respectively, than that of +36 GFP-Cre alone (FIG. 3A). These results suggest that while aurein 1.2 and citropin 1.3 both enhance the delivery of functional, non-endosomal+36 GFP-Cre protein at high concentrations, aurein 1.2 has greater efficacy than citropin 1.3 at lower concentrations.

Next, the toxicity of each fusion protein was evaluated at a range of concentrations (125 nM to 1 μM) using an ATP-dependent cell viability assay at 48 h after treatment. For +36 GFP-Cre, no cellular toxicity was observed up to 1 μM treatment, which resulted in 85% viable cells. Cells treated with 250 nM recombinant aurein 1.2-+36 GFP-Cre and citropin 1.3-+36 GFP-Cre displayed 87% and 84% viability, respectively. Applying 1 μM treatments decreased cell viability to 70% and 66%, respectively (FIG. 3B). In light of its activity and low cytotoxicity at 250 nM, the ability of aurein 1.2 to enhance cytosolic protein delivery was characterized in depth.

### Site-Directed Mutagenesis of Aurein 1.2

Aurein 1.2 (GLFDIIKKIAESF (SEQ ID NO: 5)) is a potent AMP excreted from the Australian tree frog, Litoria aurea. See, e.g., Rozek et al. Rapid Communications in Mass Spectrometry 2000, 14, 2002. Interestingly, citropin 1.3 (GLFDIIKKVASVIGGL (SEQ ID NO: 21)) is a closely related peptide and is excreted from a different Australian tree frog, Litoria citropa. See, e.g., Wegener et al. European Journal of Biochemistry/FEBS 1999, 265, 627. While the properties of aurein 1.2 have been investigated for its anti-bacterial and anti-tumorogenic abilities, its ability to enhance endosomal escape or macromolecule delivery has not been previously reported. The free peptide is thought to adopt an amphipathic alpha helical structure in solution, but the length of the helix is too short to span a lipid bilayer. See, e.g., Balla et al. European Biophysics Journal 2004, 33, 109. Therefore it has been theorized that aurein 1.2 disrupts membranes through a “carpet mechanism” in which pep-tides bind to the membrane surface in a manner that allows hydrophobic residues to interact with lipid tails and hydrophilic residues to interact with polar lipid head groups. See, e.g., Fernandez et al., Physical Chemistry Chemical Physics 2012, 14, 15739. Above a critical concentration, the peptides are thought to alter the curvature of the membrane enough to break apart the compartment.

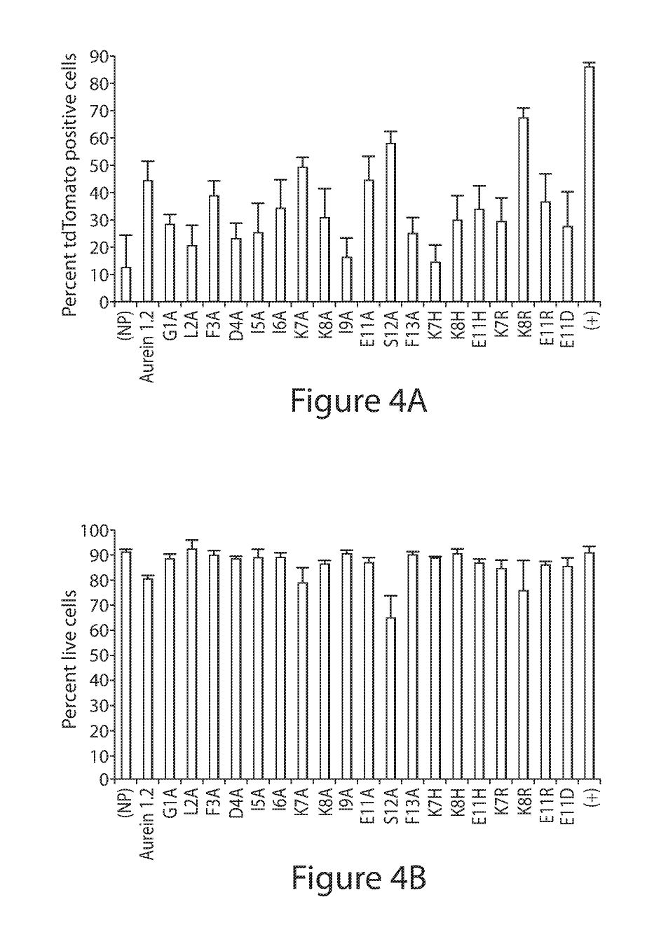

To identify the residues involved in enhancing non-endosomal protein delivery, an alanine scan of the 13 amino acid positions of aurein 1.2 was performed by cloning, expressing, and purifying each alanine mutant of aurein 1.2-+36 GFP-Cre. The resulting fusion proteins were assayed in BSR.LNL.tdTomato reporter cells as described above (Table 2). Seven positions were moderately to highly intolerant of alanine substitution. Six positions retained >70% of the activity of unmutated aurein 1.2-+36 GFP-Cre (FIG. 4A). At each of these tolerant positions, which included three positions with charged residues (K7, K8, and Ell from Table 2), we generated additional mutations in an effort to improve activity. In total, 19 mutants of aurein 1.2 were generated and tested using the Cre recombination assay. Two of the aurein variants, K8R and S12A, exhibited potentially improved overall recombination efficiency but also increased toxicity at 250 nM (FIG. 4B).

TABLE 2
Site-directed mutagenesis of aurein 1.2

| Label | Sequence | SEQ ID NO: |
|---|---|---|
| Aurein 1.2 | GLFDIIKKIAESF | 5 |
| G1A | ALFDIIKKIAESF | 37 |
| L2A | GAFDIIKKIAESF | 38 |
| F3A | GLADIIKKIAESF | 39 |
| D4A | GLFAIIKKIAESF | 40 |
| I5A | GLFDAIKKIAESF | 41 |
| I6A | GLFDIAKKIAESF | 42 |
| K7A | GLFDIIAKIAESF | 43 |
| K8A | GLFDIIKAIAESF | 44 |
| I9A | GLFDIIKKAAESF | 45 |
| E11A | GLFDIIKKIAASF | 46 |
| S12A | GLFDIIKKIAEAF | 47 |
| F13A | GLFDIIKKIAESA | 48 |
| K7H | GLFDIIHKIAESF | 49 |
| K8H | GLFDIIKHIAESF | 50 |
| E11H | GLFDIIKKIAHSF | 51 |
| K7R | GLFDIIRKIAESF | 52 |
| K8R | GLFDIIKRIAESF | 53 |
| E11R | GLFDIIKKIARSF | 54 |
| E11D | GLFDIIKKIADSF | 55 |

An alanine scan was performed on aurein 1.2 to determine positions that tolerate mutation. Charged amino acids at tolerant positions were then replaced with histidines or other charged amino acids in an attempt to increase endosomal escape efficiency. All constructs were expressed as recombinant fusion proteins with +36 GFP-Cre.

### Independent Assays of Endosomal Escape

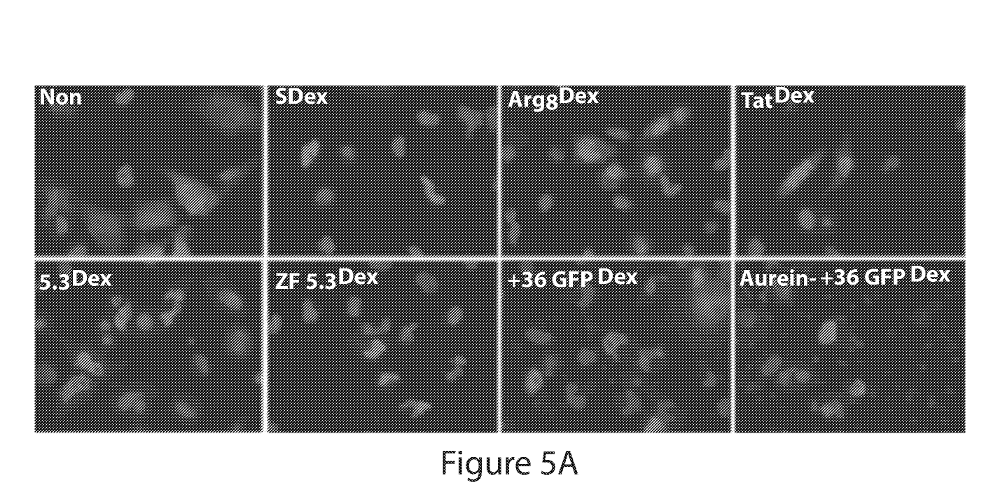

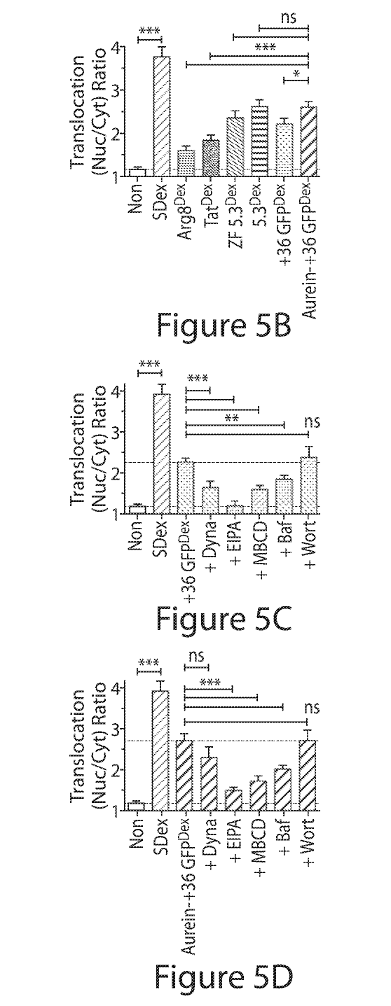

Although endosomal escape is widely considered to be the major bottleneck of cationic protein delivery, few assays quantify the ability of proteins to escape endosomes on a single-cell basis. See, e.g., Sahay et al. Nature Biotechnology 2013, 31, 653. To quantify cytosolic delivery of supercharged proteins in individual cells, a glucocorticoid receptor (GR) translocationassay described by Schepartz and colleagues was applied. See, e.g., Yu et al. Nat Biotech 2005, 23, 746; Holub et al. Biochemistry 2013, 52, 9036. In untreated HeLa cells expressing mCherry-labeled GR (GR-mCherry), the GR distributes nearly uniformly throughout the cell interior, resulting in a nuclear-to-cytoplasm translocation ratio (TR) of 1.17 (FIGS. 5A and 5B). Upon treatment with the cell-permeable glucocorticoid dexamethasone-21-thiopropionic acid (SDex) at a concentration of 1 μM for 30 min, GR-mCherry relocates almost exclusively to the nucleus, resulting in a TR of 3.77 (FIGS. 5A and 5B).

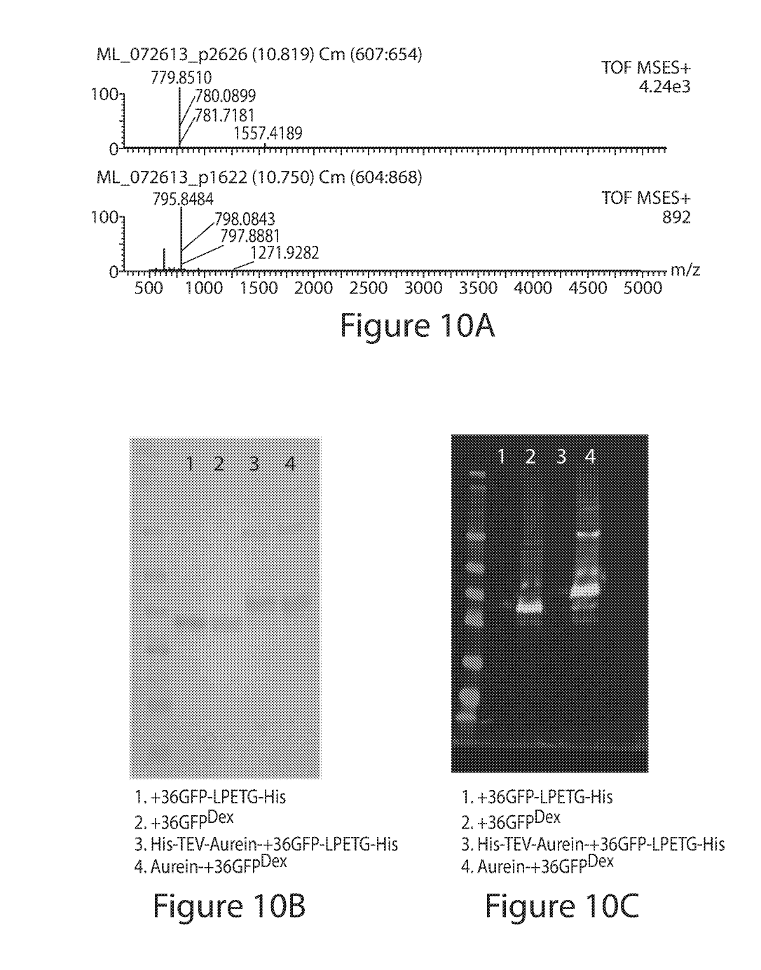

Dexamethasone conjugates of +36 GFP (+36 GFP^{Dex}) and aurein 1.2-+36 GFP (aurein 1.2-+36 GFP^{DCX}) were generated via sortase-mediated conjugation (FIG. 10). Conjugated to these proteins, SDex is no longer cell permeable and cannot activate the GR for nuclear translocation unless the protein-SDex conjugate can access the cytosol. Treatment of HeLa cells expressing GR-mCherry with 1 μM aurein 1.2-+36 GFP^{Dex} for 30 min resulted in a TR of 2.62, which was significantly greater (p<0.05) than that of +36 GFP^{Dex} (TR=2.23). As positive controls, these cells were treated with canonical cell permeable peptides (Tat^{Dex} and Arg₈^{Dex}) and miniature proteins containing a penta-Arg motif that reach the cytosol intact, with efficiencies exceeding 50% (5.3^{Dex} and ZF 5.3^{Dex}). See, e.g., LaRochelle et al. Journal of the American Chemical Society 2015, 137, 2536. Aurein 1.2-+36 GFP^{Dex} (TR=2.62), activated significantly greater levels of GR-mCherry translocation (p<0.001) than Tat^{Dex} (TR=1.87) and Arg8^{Dex} (TR=1.63) and similar levels evoked by miniature proteins 5.3^{Dex} (TR=2.62) and ZF 5.3^{Dex} (TR=2.38) (FIGS. 5A and 5B). Taken together, these results suggest that aurein 1.2-+36 GFP^{Dex} exhibits an improved ability to access the cytoplasm over +36 GFP^{Dex} and canonical cell permeable peptides.

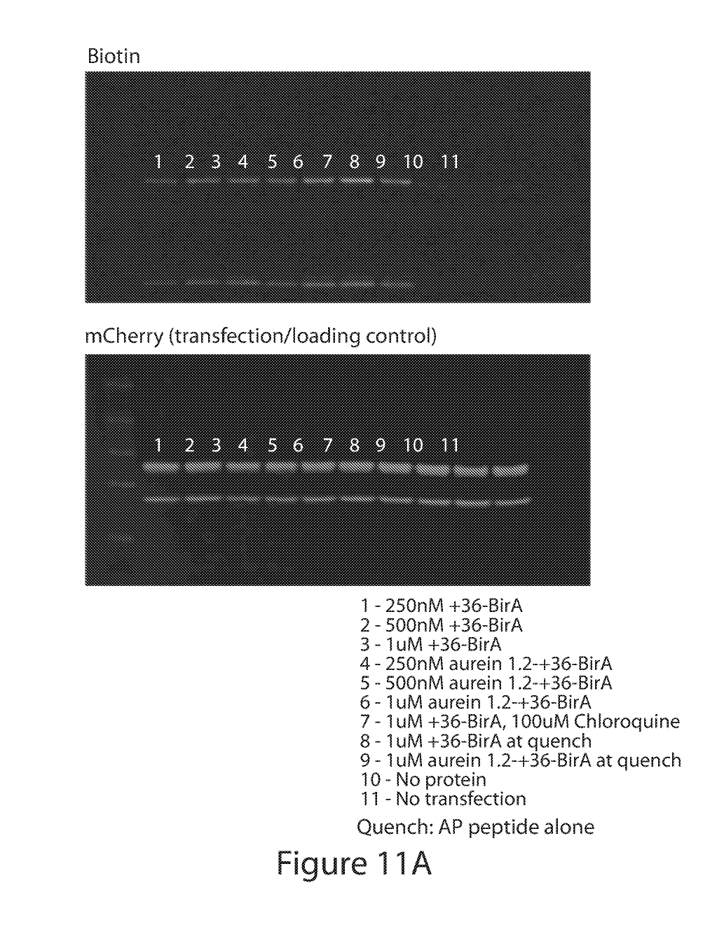

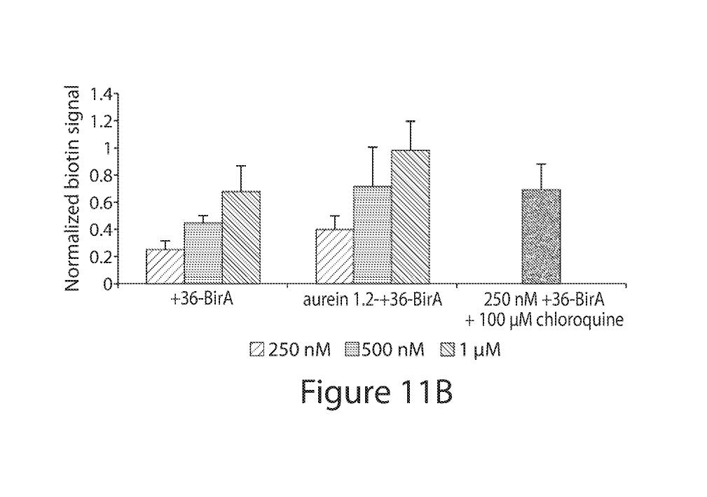

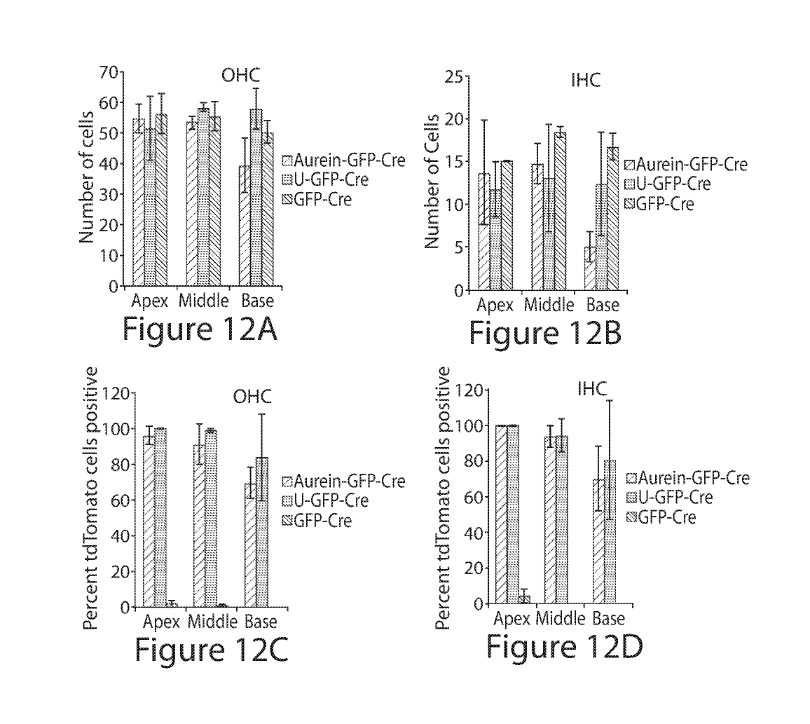

As an additional, independent assay of non-endosomal protein delivery, the ability of aurein 1.2 to enhance the non-endosomal delivery of an evolved biotin ligase (BirA) enzyme was tested using the method developed by Ting and coworkers. See, e.g., Howarth et al. Nature protocols 2008, 3, 534. BirA catalyzes the biotinylation of a 15-amino acid acceptor peptide (AP). We transfected a mCherry-AP fusion plasmid into HeLa cells. Biotinylation of mCherry can only occur in the presence of cytosolic BirA. To assess the non-endosomal delivery of +36 GFP-BirA protein, mCherry-AP biotinylation was quantified by (FIG. 11A). Treatment with 250 nM aurein 1.2-+36 GFP-BirA resulted in a 50% increase in biotinylation signal compared with 250 nM of +36 GFP-BirA alone (FIG. 11B). We also observed a dose-dependent increase in AP-biotinylation across treatment concentrations (250 nM, 500 nM, and 1 μM) for both aurein 1.2-(+36 GFP)-BirA and unfused+36 GFP-BirA constructs. These results are consistent with the results of the GR translocation assay, and further suggest that aurein 1.2 enhances the endosomal escape of superpositively charged proteins.

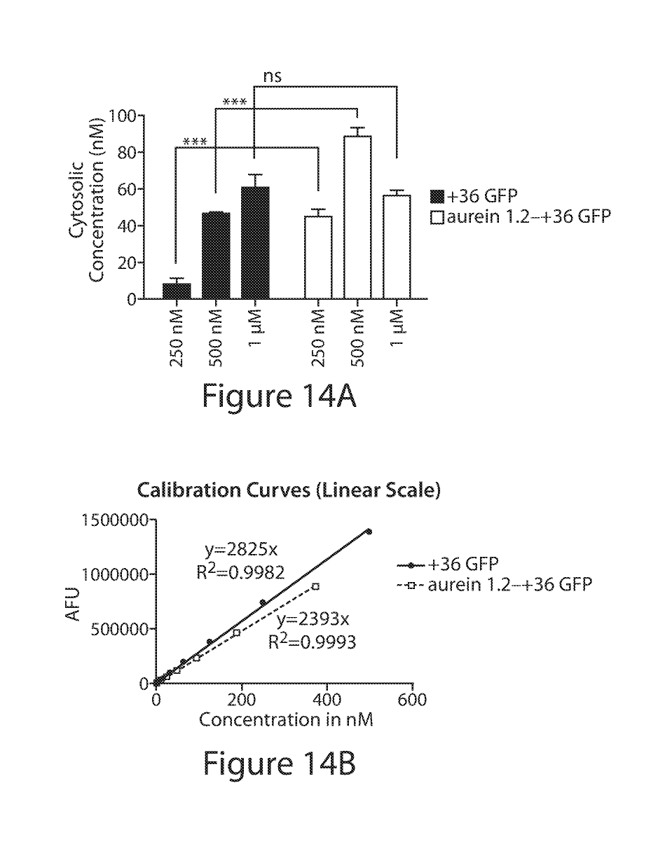

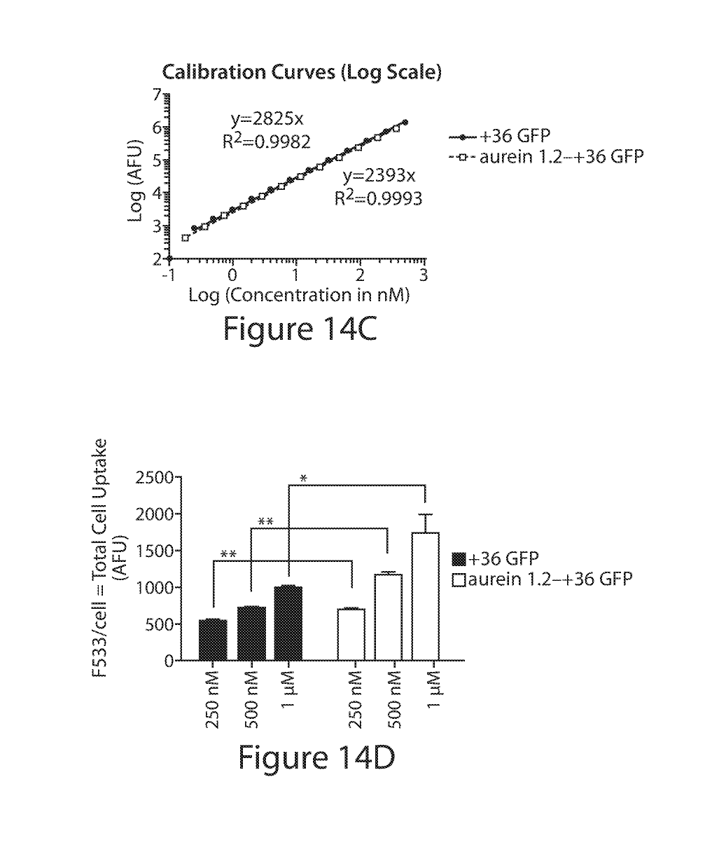

In order to directly quantify the increase in non-endosomal delivery resulting from aurein 1.2, a cytosolic fractionation experiment was performed to calculate the cytosolic concentrations of delivered protein. HeLa cells were treated with +36 GFP or aurein 1.2-+36 GFP at 250 nM, 500 nM, and 1 μM. After 30 min of treatment, cells were washed, homogenized, and fractionated by ultracentrifugation. The cytosolic concentration of delivered protein was calculated from the GFP fluorescence of the cytosolic fraction together with a standard curve relating fluorescence to known concentrations of +36 GFP and aurein 1.2-+36 GFP added to cytosolic extract (FIGS. 14B and 14C). At 250 nM, treatment with aurein 1.2-+36 GFP resulted in ˜5-fold more delivered cytosolic protein than treatment with +36 GFP alone (FIG. 14C). This difference decreased with increasing protein concentration, likely due to the influence of alternate uptake pathways or delivery bottlenecks at high protein concentrations. In contrast, the total amount of aurein 1.2-+36 GFP versus +36 GFP uptaken by cells was similar, with aurein 1.2-+36 GFP showing 1.3-fold higher total cellular uptake at 250 nM. These results directly demonstrate that aurein 1.2 increases the cytosolic concentration of cationic proteins that enter cells predominantly through endosomes, and are consistent with the above findings that aurein 1.2 has the greatest effect on enhancing non-endosomal delivery at ˜250 nM (FIG. 3A).

### Effect of Endocytic Inhibitors on +36 GFP and Aurein 1.2-+36 GFP Delivery

Endocytosis plays a key role in the cytosolic delivery of superpositively charged proteins¹⁸. To probe the role of endocytosis in the delivery of supercharged proteins with or without aurein 1.2, we treated cells expressing GR-mCherry with either+36 GFPDex or aurein 1.2-+36 GFPDex in the presence of known endocytic inhibitors. The cortical actin remodeling inhibitor N-ethyl-isopropyl amiloride (EIPA), the cholesterol-sequestering agent methyl-P-cyclodextrin (MBCD), and the endosomal vesicular ATPase inhibitor bafilomycin (Baf) all strongly reduced the ability of both proteins to stimulate GR-mCherry translocation. Blocking maturation of Rab5+ vesicles by treatment with the phosphatidylinositol 3-kinase inhibitor wortmannin (Wort) did not influence reporter translocation of either+36 GFPDex or aurein 1.2-+36 GFPDex (FIGS. 5C and 5D). In contrast, treatment with the small-molecule dynamin II inhibitor Dynasore (Dyna) significantly suppressed the ability of +36 GFPDex to stimulate GR-mCherry translocation (TR=1.64) (FIG. 5C) but had little influence on the cytosolic delivery of aurein 1.2-+36 GFPDex (TR=2.30) (FIG. 5D). Taken together, these results suggest that active endocytosis is required for uptake of +36 GFP and aurein 1.2-+36GFP into the cell interior, and that the two proteins may traffic differently into the cell interior.

### Aurein 1.2 can Greatly Increase Protein Delivery Efficiency In Vivo

To evaluate the ability of aurein 1.2 to increase the efficacy of cationic protein delivery in vivo, proteins were delivered to the inner ear of Cre reporter transgenic mice that express tdTomato upon Cre-mediated recombination. This animal model was chosen due to its confined injection volume, the presence of well-characterized cell types, and the existence of genetic deafness models that would facilitate future studies of protein delivery to treat hearing loss. It was previously demonstrated that+36 GFP-Cre alone can be delivered to mouse retina, albeit resulting in only modest levels of recombination consistent with inefficient endosomal escape.

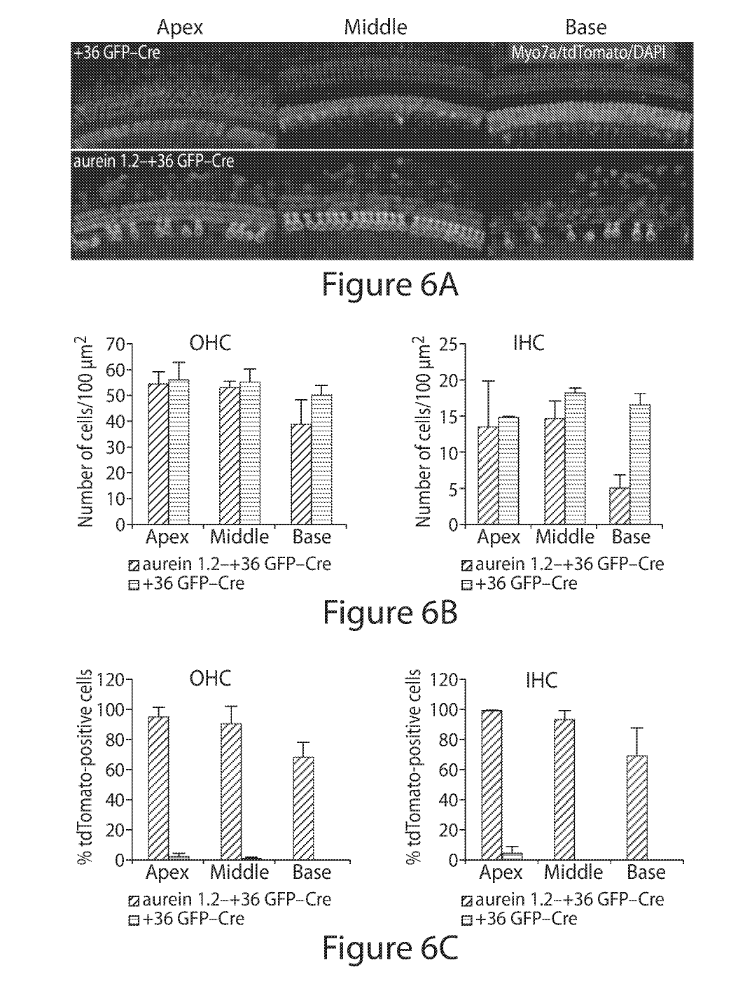

Anesthetized postnatal day 2 (P2) mice were injected with 0.4 μL of 50 μM+36 GFP-Cre or aurein 1.2-+36 GFP-Cre solutions in the scala media to access the cochlear cells. Five days after injection, the cochleas were harvested for immunolabeling of inner ear cell markers and imaging for tdTomato florescence (FIG. 6A). Both the hair cells (Myo7a+) and supporting cells (Myo7a−) were evaluated for td Tomato signal. The total number of hair cells and supporting cells (by DAPI labeling) in the sensory epithelium (SE) was used to determine the relative toxicity of aurein 1.2-+36 GFP-Cre to the baseline treatment of +36 GFP-Cre (FIG. 6A). Overall, an average of 96%, 92% and 66% of cochlear cells survived aurein 1.2-+36 GFP-Cre treatment as compared to +36 GFP-Cre treatment in the apex, middle, and base tissue samples, respectively (FIG. 6A). +36 GFP-Cre treatment resulted in low levels of recombination only in inner hair cells (IHC) of the apex of the cochlea (4.4%) but not in the middle or base of the cochlear hair cells or any cochlear supporting cells. In contrast, treatment with aurein 1.2-+36 GFP-Cre resulted in very high Cre-mediated recombination levels throughout the apex, middle, and base samples of outer hair cells (OHC) (96%, 91%, and 69%, respectively), inner hair cells (100%, 94%, and 70%, respectively), as well as supporting cells (arrows) (FIGS. 6A and 6C).

The observed levels of recombination in the inner hair cells from aurein 1.2-+36 GFP-Cre are comparable to that of adeno-associated virus type 1 (AAV1) gene transfection. For outer hair cells, we have previously shown similar levels of recombination using liposome-mediated delivery of supernegatively-charged GFP-Cre. The aurein 1.2-+36 GFP-Cre delivery system is the only method that showed significant recombination levels in both inner and outer hair cells, and does not require any virus or other molecules beyond a single polypeptide. Significantly, aurein 1.2-+36 GFP-Cre also extended delivered recombinase activity to additional cochlear supporting cells. These results suggest aurein 1.2-+36 GFP-Cre delivery system to be a promising method for in vivo protein delivery into both hair cells and supporting cells of the inner ear. See, e.g., Akil et al. Neuron 2012, 75, 283; Zuris et al. Nat Biotech 2015, 33, 73; Taura et al. Neuroscience 2010, 166, 1185; Izumikawa et al. Nature Medicine 2005, 11, 271.

As demonstrated in this Example, a 13-residue peptide, aurein 1.2, and derivates thereof can increase the efficiency of non-endosomal protein delivery by screening a panel of known membrane-active peptides. The results from a small screen of 22 peptides are consistent with the hypothesis that some peptides can selectively disrupt the endosomal membrane without disrupting the mammalian cell membrane. The effectiveness of aurein 1.2 and derivatives thereof is highly dependent on their sequences, as several other closely related peptides did not enhance protein delivery (Tables 1 and 2). Notable endosomal escape peptides include those with amino acid sequences set forth in SEQ ID NOs: 5, 8, 19, 21, 23, 39, 43, 47, and 53. Subtle differences in amino acid composition led to dramatic changes in membrane activity among peptides tested, highlighting the difficulty of rationally designing peptides that enhance non-endosomal delivery. Moreover, the lack of correspondence between peptide cationic charge and non-endosomal delivery efficiency suggests that aurein 1.2 does not enhance non-edosomal delivery simply by promoting endocytosis. While none of the tested variants of aurein 1.2 substantially outperformed the original peptide, we identified several amino acids that could be altered without loss of activity. These findings also provide a starting point for further optimization to discover next-generation endosomolytic peptides with improved efficacy and reduced toxicity.

Three independent assays for non-endosomal protein delivery (Cre recombination, GR translocation, and BirA activity on a cytoplasmic peptide), together with the peptide mutational studies described above, all suggest that aurein 1.2-fusion enhances endosomal escape of superpositively charged proteins. Moreover, these assays collectively demonstrated the ability of aurein 1.2 to mediate the non-endosomal delivery of +36 GFP fused to different proteins (or small molecules), suggesting that aurein 1.2 facilities endosomal escape in a manner that is at least somewhat cargo-independent.

The in vivo protein delivery experiments described above revealed dramatic increases in non-endosomal functional Cre recombinase delivery into the diverse inner ear cell types including hair cells and supporting cells of live mice upon fusion with aurein 1.2. Indeed, aurein 1.2-fused+36 GFP-Cre construct resulted in highly efficient recombination levels across the main cochlear sensory epithelial cell classes studied in this work, all but one of which were unaffected by +36 GFP-Cre treatment. Taken together, these results suggest that aurein 1.2 is a 13-residue, potent, genetically encodable, endosome escape-enhancing peptide that can greatly increase the efficiency of non-endosomal protein delivery in vitro and in vivo without requiring the use of additional components beyond the protein of interest.

### Materials and Methods

### Construction of Expression Plasmids

Sequences of all constructs used in this paper are listed below. All protein constructs were generated from previously reported plasmids for protein of interest cloned into a pET29a expression plasmid. See, e.g., Thompson et al. In Methods in Enzymology; Wittrup et al., Eds.; Academic Press: 2012; Volume 503, p 293. All plasmid constructs generated in this work will be deposited with Addgene.

### Expression and Purification of Proteins

E. coli BL21 STAR (DE3) competent cells (Life Technologies) were transformed with pET29a expression plasmids. Colonies from the resulting expression strain was directly inoculated in 1 L of Luria-Bertani (LB) broth containing 100 μg/mL of ampicillin at 37° C. to OD₆₀₀=-1.0. Isopropyl β-D-1-thiogalactopyranoside (IPTG) was added at 0.5 mM to induce expression and the culture was moved to 20° C. After ˜16 hours, the cells were collected by centrifugation at 6,000 g and resuspended in lysis buffer (Phosphate buffered saline (PBS) with 1 M NaCl). The cells were lysed by sonication (1 sec pulse-on, 1 sec pulse-off for 6 min, twice, at 6 W output) and the soluble lysate was obtained by centrifugation at 10,000 g for 30 minutes.

The cell lysate was incubated with His-Pur nickel-nitriloacetic acid (Ni-NTA) resin (Thermo Scientific) at 4° C. for 45 minutes to capture His-tagged protein. The resin was transferred to a 20-mL column and washed with 20 column volumes of lysis buffer plus 50 mM imidazole. Protein was eluted in lysis buffer with 500 mM imidazole, and concentrated by Amicon ultra centrifugal filter (Millipore, 30-kDa molecular weight cut-off) to ˜50 mg/mL. The eluent was injected into a 1 mL HiTrap SP HP column (GE Healthcare) after dilution into PBS (5-fold). Protein was eluted with PBS containing a linear NaCl gradient from 0.1 M to 1 M over five column volumes. The eluted fractions containing protein were concentrated to 50 μM as quantified by absorbance at 488 nm assuming an extinction coefficient of 8.33×10⁴ M⁻¹cm⁻¹ as previously determined, snap-frozen in liquid nitrogen, and stored in aliquots at −80° C.

### Cell Culture

All cells were cultured in Dulbecco's modification of Eagle's medium (DMEM w/glutamine, Gibco) with 10% fetal bovine serum (FBS, Gibco), 5 I.U. penicillin, and 5 g/mL streptamycin. All cells were cultured at 37° C. with 5% CO₂.

### Peptide Synthesis

Peptides were ordered from ChinaPeptides Co., LTD, each 4 mg, purity >90%. HPLC and MALDI data were provided with lyophilized peptides. Peptides were resuspeneded in DMSO to a final concentration of 10 mM.

### Sortase Conjugation

All reactions were performed in 100 mM Tris buffer (pH 7.5) with 5 mM CaCl₂ and 1 M NaCl. For peptide conjugation to the N-terminus of GGG-+36-GFP, 20 μM of protein with N-terminal Gly-Gly-Gly was incubated with 400 μM of peptide with C-terminal LPETGG (SEQ ID NO: 91) and 1 μM eSrtA for 2 hours at room temperature in a 50 μL reaction. The unreacted peptides were removed through spin filtration with an Amicon Ultra-0.5 Centrifugal Filter Unit (Millipore, 30-kDa molecular weight cut-off). The reaction mixture was washed twice with 500 μL of buffer each time to a final concentration of 50 μL. Conjugation efficiency was determined through LC-MS (Agilent 6220 ESI-TOF) using protein deconvolution through MaxEnt (Waters) by comparing relative peak intensities.

For conjugation of GGGK^{Dex} (SEQ ID NO: 100) to +36-GFP-LPETG (SEQ ID NO: 90)-His₆, 10 μM of protein was incubated with 400 μM of peptide and 2 μM eSrtA at room temperature. The reaction was quenched with 10 mM ethylenediaminetetraacetic acid (EDTA) after 2 hours. For aurein 1.2-+36-GFP-LPETG (SEQ ID NO: 90)-His₆, a N-terminal His₆-ENLYFQ (SEQ ID NO: 99) was added to prevent sortase reaction with the N-terminal glycine of aurein 1.2. The N-terminal tag was removed with 200 μM TEV protease at 4° C. for 16 hours to release the native N-terminal sequence of aurein 1.2-+36-GFP. Successful conjugation of GGGK^{Dex} (SEQ ID NO: 100) removes the C-terminal His₆ tag and allows for purification through reverse Ni-NTA column. Unreacted protein binds to the Ni-NTA, and the unbound protein was collected and concentrated as described above.

### Plasmid Transfection

Plasmid DNA was transfected using Lipofectamine 2000 (Life Technologies) according the manufacturer's protocol.

### Synthesis of Dexamethasone-21 Thiopropionic Acid (SDex)

Synthesis of dexamethasone-21-mesylate was performed as previously described. See, e.g., Simons et al. J Org Chem 1980, 45, 3084; Dunkerton et al. Steroids 1982, 39, 1.2 g of dexamethasone stirring in 38 mL anhydrous pyridine under nitrogen was reacted with 467.2 μg methanesulfonyl chloride (1.2 eq.) on ice for 1 hour, after which another 311 μg methanesulfonyl chloride was added and allowed to react overnight (16 hours) on ice. Next, the reaction was added to 800 mL of ice water and dexamethasone-21-Mesylate (Dex-21-OMs) formed a white precipitate. The slurry was filtered and the precipitate washed with 800 mL of ice water, dried under high vacuum overnight and quantified by LC-MS (m/z 471.19 Da, 83% yield).

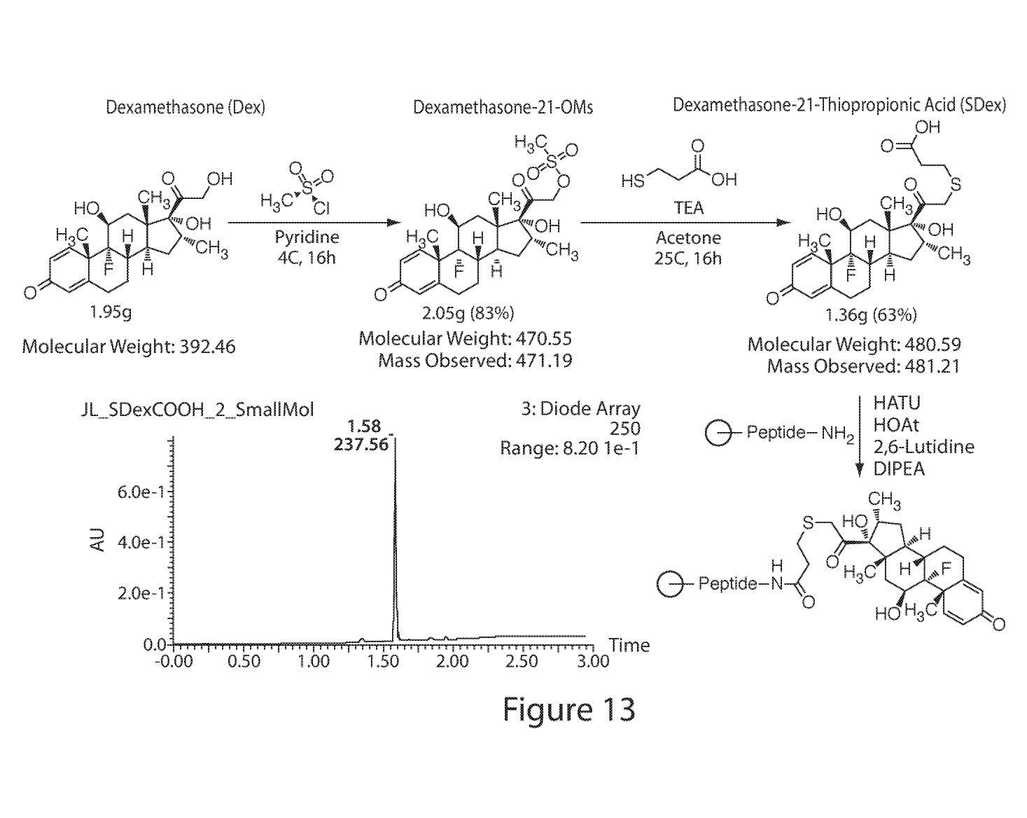

Dexamethasone-21-thiopopionic acid (SDex) was prepared as previously described. See, e.g., Kwon, et al. J Am Chem Soc 2007, 129, 1508. 2.05 g of Dex-21-OMs was added to 2 eq. thiopropionic acid and 4 eq. triethylamine stirring in anhydrous acetone at room temperature overnight. The following morning, the reaction was added to 800 mL of ice water and acidified with 1 N HCl until SDex, visible as an off-white solid, precipitation was complete. The mixture was filtered, washed with 800 mL ice cold water acidified to pH 1 with HCl, dried under high vacuum overnight and analyzed by LC-MS (m/z 481.21 Da, 63% yield) (FIG. 13).

### Synthesis and Purification of GGGK^{Dex}(SEQ ID NO: 100)

GGGK^{Dex} (SEQ ID NO: 100) was synthesized on Fmoc-Lys (Mtt)-Wang resin (BACHEM, D-2565) using microwave acceleratin (MARS, CEM). Coupling reactions were performed using 5 equivalents of Fmoc-Gly-OH (Novabiochem, 29022-11-5), 5 equivalents of PyClock (Novabiochem, 893413-42-8) and 10 equivalents of diisopropylethylamine (DIEA) in N-methylpyrrolidone (NMP). Fmoc groups were removed using 25% piperidine in NMP (efficiency quantified; ε₂₉₉=6234 M⁻¹cm⁻¹ in acetonitrile) and Mtt groups were removed by incubating the Fmoc-GGGK(SEQ ID NO: 100) (Mtt)-resin with 2% trifluoroacetic acid (TFA) in dichloromethane (DCM) for 20 min, after which the resin was washed with 2% TFA in DCM until the characteristic yellow color emitting from the Mttcation subsided. After Mtt removal, SDex-COOH (Dex-21-thiopropinonic acid) was coupled to the NE of the lysine side-chain by incubating the Fmoc-GGGK (SEQ ID NO: 100)-resin with 2.5 eq. SDex-COOH, 2.5 eq. HATU, 2.5 eq. HOAt, 5 eq. DIEA and 5 eq. 2,6-lutidine in 2.5 mL NMP overnight, at room temperature, on an orbital shaker. After SDex-labeling, Fmoc-GGGK^{Dex} (SEQ ID NO: 100)-resin was washed thoroughly with NMP and DCM, the N-terminal Fmoc was removed using 25% piperidine in NMP, and crude peptides were dissociated from the resin by incubating the GGGK^{Dex} (SEQ ID NO: 100)-resin in a cleavage cocktail composed of 81.5% trifluoroacetic acid (TFA), 5% thioanisole, 5% phenol, 5% water, 2.5% ethanedithiol and 1% triisopropylsilane for 30 min at 38° C. Crude peptides were precipitated in 40 mL cold diethyl ether, resuspended in water, lyophilized and purified via reverse phase high-pressure liquid chromatography (HPLC) using a linear gradient of acetonitrile and water with 0.1% TFA across a C18 (VYDAC, 250 mm×10 mm ID) column. Purified peptides were lyophilized and stored at 4° C. Polypeptide identity was confirmed by mass spectrometry on a Waters QToF LC-MS, and purity was measured by analytical reverse-phase HPLC (Shimadzu Instruments) using a C18 column (Poroshell 120 SB-C18, 2.7 μm, 100 mm×3 mm ID, Agilent).

### Image Processing for Primary Screen

BSR.LNL.tdTomato cells were plated at 10,000 cells per well in black 384-well plates (Aurora Biotechnologies). Cells were treated with Cre fusion proteins diluted in serum-free DMEM 24 hours after plating and incubated for 4 hours at 37° C. Following incubation, the cells were washed three times with PBS+20 U/mL heparin. The cells were incubated a further 48 h in serum-containing media. Cells were fixed in 3% paraformaldehyde and stained with Hoescht 33342 nuclear dye. Images were acquired on an ImageXpress Micro automated microscope (Molecular Devices) using a 4× objective (binning 2, gain 2), with laser- and image-based focusing (offset −130 μm, range ±50 μm, step 25 μm). Images were exposed for 10 ms in the DAPI channel (Hoechst) and 500 ms in the dsRed channel (tdTomato). Image analysis was performed using the cell-scoring module of MetaXpress software (Molecular Devices). All nuclei were detected with a minimum width of 1 pixel, maximum width of 3 pixels, and an intensity of 200 gray levels above background. Positive cells were evaluated for uniform signal in the dsRed channel (minimum width of 5 pixels, maximum width of 30 pixels, intensity >200 gray levels above background, 10 μm minimum stained area). In total, nine images were captured and analyzed per well, and 16 wells were treated with the same fusion protein. The primary screen was completed in biological triplicate.

### Cre Delivery Assay

Uptake and delivery assays for Cre fusion proteins were performed as previously described. Briefly, proteins were diluted in serum-free DMEM and incubated on the cells in 48-well plates for 4 hours at 37° C. Following incubation, the cells were washed three times with PBS+20 U/mL heparin. The cells were incubated a further 48 hours in serum-containing media prior to trypsinization and analysis by flow cytometry. All flow cytometry were carried out on a BD Fortessa flow cytometer (Becton-Dickinson) using 530/30 nm and 610/20 nm filter sets. Toxicity for aurein 1.2 and citropin 1.3 validation assays was determined using CellTiterGlo assay (Promega) in 96-well plates following manufacturer protocol. Toxicity for alanine scan mutational analysis was determined with LIVE/DEAD fixable far-red dead cell stain (Life Technologies) with 635 nm laser and 670/30 nm filter.

GR-mCherry translocation assay

One day prior to transfection 10,000 HeLa cells in 200 μL of DMEM (10% FBS, lx PenStrep) were plated into single wells of a 96-well MatriCal glass bottom microplate (MGB096-1-2-LG-L) and allowed to adhere overnight. The following day, cells were transfected with GR-mCherry using Lipofectamine® 2000 technologies. Following transfection, cells were allowed to recover overnight in DMEM (+10% FBS). The following day, cells were treated with dexamethasone (Dex) or 1 μM Dex-protein conjugate in the presence or absence of inhibitor diluted into DMEM (without phenol red, +300 nM Hoescht33342). Following one hour treatment, cells were washed twice with 200 μL of HEPES-Krebs-Ringer's (HKR) buffer (140 mM NaCl, 2 mM KCl, 1 mM CaCl₂, 1 mM MgCl₂, and 10 mM HEPES at pH 7.4), after which 100 μL of HKR buffer was overlaid onto the cells and images were acquired on a Zeiss Axiovert 200M epifluorescence microscope outfitted with Ziess AxiocammRM camera and an EXFO-Excite series 120 Hg arc lamp. The translocation ratio (the ratio of median GFP intensity in the nuclear and surrounding regions) for individual cells was measured using CellProfiler® as described. To examine the effect of endocytosis inhibitors, HeLa cells were pretreated for 30 min with DMEM (without phenol red) containing inhibitors (80 μM Dynasore, 5 mM MBCD, 50 μM EIPA, 200 nM bafilomycin or 200 nM wortmannin) at 37° C. for 30 minutes before incubation with Dex or Dex-protein conjugates.

### BirA Translocation Assay

One day prior to transfection, 100,000 HeLa cells in 1 mL of DMEM (10% FBS, lx PenStrep) were plated into single wells of a 12-well tissue culture plate and allowed to adhere overnight. Cells were transfected with mCherry-AP fusion protein using Lipofectamine® 2000 technologies according to manufacture guidelines24 h before protein treatment. Next day, transfected cells were treated for 1 hour at 37° C. with +36 GFP-BirA or aurein 1.2-+36 GFP-BirA diluted in serum-free DMEM at 250 nM, 500 nM and 1 μM concentrations. 250 nM+36 GFP-BirA+100 μM chloroquine was also used as a positive control for endosomal escape. The cells were washed three times with PBS containing heparin to remove excess supercharged proteins that were not internalized. The cells were then treated with 100 μL of 10 μM biotin and 1 mM ATP in PBS for 10 min. The reaction was quenched with excess (10 μL of 8 mM) synthesized AP before cells were trypsinized and lysed. To verify that extracellular BirA was not generating signal during lysis, 1 μM+36 GFP-BirA or aurein 1.2-+36 GFP-BirA was added during the quench step to untreated wells. Cells were lysed with 100 μL of trypsin and lysed with QlAshredder columns (Qiagen). 30 μL of lysate was loaded onto 4-12% Bis-Tris Bolt gels in Bolt-MES buffer (Life Technologies) and ran for 20 min at 200 volts. Gels were transferred to PVDF membrane using iBlot2 transfer system (Life Technologies). Biotinylation was measured through western blotting using the LI-COR quantitative infrared fluorescent antibodies and the Odyssey Imager detection system. To normalize for transfection and gel loading variables, the ratio of biotin signal to mCherry signal was used for comparison.

### Cytosolic Fractionation Assay

One day prior to fractionation, 4×10⁶ HeLa cells were plated in 20 mL of DMEM (10% FBS, lx PenStrep, no phenol red) in 175-cm² culture flasks and allowed to adhere for 15 hours. The following day, the media was removed from each flask and the cells were washed twice with clear DMEM (no FBS, no PenStrep, no phenol red). The media was replaced with 7 mL of clear DMEM containing+36 GFP or aurein 1.2-+36 GFP at a concentration of 250 nM, 500 nM, or 1 μM. Several flasks were treated with clear DMEM to be used as negative controls and to generate calibration curves with the cytosolic extracts. The cells were incubated for 30 min at 37° C., 5% CO₂ after which they were washed three times with PBS. Using a cell-scraper, the cells were suspended in 5 mL of PBS, transferred into a 15 mL Falcon tube, and pelleted at 500 g for 3 min. The cells were resuspended in 1 mL PBS, counted using an automated cell counter (Auto T4, Cellometer®), and pelleted again at 500 g for 3 min. The cell pellet was resuspended in ice-cold isotonic sucrose (290 mM sucrose, 10 mM imidazole, pH 7.0 with 1 mM DTT, and cOmplete™, EDTA-free protease inhibitor cocktail) and transferred to a glass test tube on ice. The cells were homogenized with an Omni TH homogenizer outfitted with a stainless steel 5 mm probe for three 30 s pulses on ice with 30 s pauses between the pulses. The homogenized cell lysate was sedimented at 350 Kg in an ultracentrifuge (TL-100; Beckman Coulter) for 30 min at 4° C. using a TLA 120.2 rotor. The supernatant (cytosolic fraction) was analyzed in a 96-well plate on a fluorescence plate reader (Synergy 2, BioTek, excitation=485+/−10 nm, emission=528+/−10 nm). The concentration of the protein conjugate in the cytosol was determined using a standard curve relating fluorescence to known protein concentrations. To generate the standard curve, known concentrations of +36 GFP and aurein 1.2-+36 GFP between 0.2 nM and 1 μM were added to cytosolic extracts of the untreated negative controls. For background subtraction, several wells containing cytosolic extracts from untreated cells were averaged, and this average was subtracted from each well.

### Total Protein Delivery Assay

One day prior to the experiment, 100,000 HeLa cells/well were plated in DMEM (10% FBS, lx PenStrep, no phenol red) in 6-well plates and allowed to adhere for 15 hours. The following day, the media was removed from each well and the cells were washed twice with clear DMEM (no FBS, no PenStrep, no phenol red). The media was replaced with 1 mL of clear DMEM containing+36 GFP or aurein 1.2-+36 GFP at concentrations of 250 nM, 500 nM, or 1 μM. The cells were incubated for 30 min at 37° C., 5% CO₂ after which they were washed three times with PBS containing 20 U/mL heparin (Sigma) to remove surface-bound cationic protein. The cells were trypsinized for 5 min, pelleted in serum-containing DMEM for 3 min at 500 g, washed with 1 mL PBS, and pelleted again for 3 min at 500 g. The cell pellet was resuspended in 100 μL PBS. Flow cytometry was performed on a BD Accuri C6 Flow Cytometer at 25° C. Cells were analyzed in PBS (excitation laser=488 nm, emission filter=533+/−30 nm). At least 10,000 cells were analyzed for each sample. For background subtraction, wells were treated with clear DMEM only. The average of three untreated wells was subtracted from each+36 GFP conjugate-containing well.

### Microinjection of Proteins to Mouse Inner Ear

P1-2 Gt(ROSA)26Sor^{tm14(CAG-tdTomato)Hze} mice were used for aurein 1.2-+36-GFP-Cre and +36-GFP-Cre injection. The Rosa26-tdTomato mice were from the Jackson Laboratory. Animals were used under protocols approved by the Massachusetts Eye & Ear Infirmary IACUC committee. Mice were anesthetized by hypothermia on ice. Cochleostomies were performed by making an incision behind the ear to expose the cochlea. Glass micropipettes held by a micromanipulator were used to deliver the complex into the scala media, which allows access to inner ear hair cells. The total delivery volume for every injection was 0.4 μL per cochlea and the release was controlled by a micromanipulator at the speed of 69 nL/min.

### Immunohistochemistry and Quantification

5 days after injection, the mice were sacrificed and cochlea were harvested by standard protocols. See, e.g., Sage et al. Science 2005, 307, 1114. For immunohistochemistry, antibodies against hair-cell markers (Myo7a) and supporting cells (Sox2) were used following a previously described protocol. To quantify the number of tdTomato positive cells after aurein 1.2-+36-GFP-Cre and +36-GFP-Cre, we counted the total number of inner and outer hair cells in a region spanning 100 μm in the apex, middle, and base turn of the cochlea.

Determining the Efficacy of Non-Endosomal Delivery with Aurein 1.2 in Trans

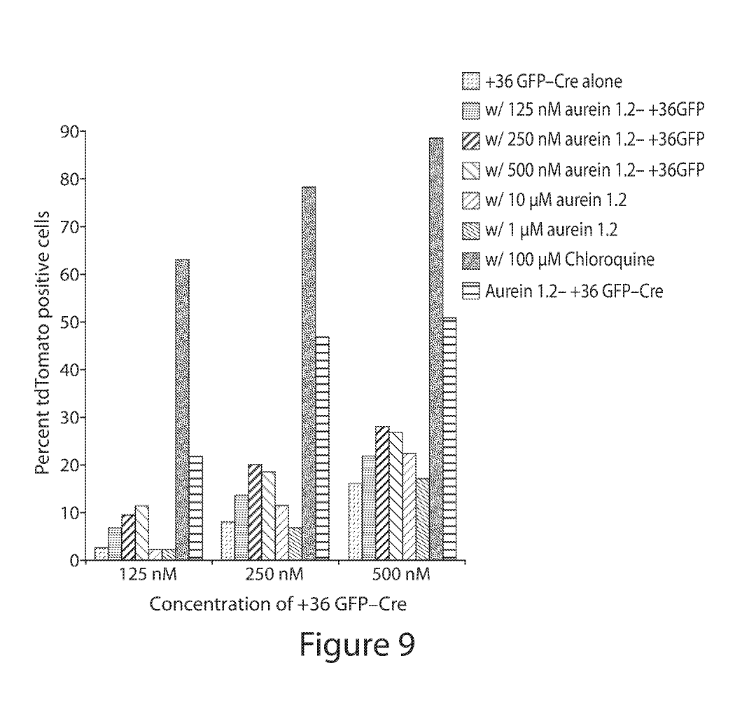

Although the primary screen was performed with aurein 1.2 conjugated to +36 GFP-Cre, it is possible that aurein potentiates non-endosomal delivery through trans-acting mechanisms. To test this possibility, we assayed functional Cre recombinase delivery of +36 GFP-Cre mixed with aurein 1.2, or mixed with aurein 1.2-+36 GFP fusion protein lacking Cre at various concentrations (FIG. 9). Aurein 1.2 when added in trans did not affect the functional delivery of +36 GFP-Cre, consistent with a model in which aurein 1.2 must be endocytosed in order to increase delivery potency. In contrast, adding aurein 1.2-+36 GFP to +36 GFP-Cre increased non-endosomal delivery potency in a dose-dependent manner (FIG. 9), albeit less potently than that of the aurein 1.2-+36 GFP-Cre fusion protein. This result supports a model in which endosomes containing both aurein 1.2-+36 GFP and +36 GFP-Cre release protein cargo more efficiently than endosomes lacking aurein 1.2 since the number of endosomes containing both proteins when administered in trans is dependent on the concentration of both proteins. Table 3 below shows peptide sequence and primers for the alanine scan of aurein 1.2.

TABLE 3
Peptide sequence and primers for alanine scan of aurein 1.2

| | SEQ ID NO: | Sequence | SEQ ID NO: | Primers |
|---|---|---|---|---|
| Aurein 1.2 | 5 | GLFDIIKKIAESF | 56 | ggcctgtttgatattattaaaaaaattgcggaaagcttt |
| Aurein 1 | 37 | [symbol] LFDIIKKIAESF | 57 | [symbol] ctgtttgatattattaaaaaaattgcggaaagcttt |
| Aurein 2 | 38 | G [symbol] FDIIKKIAESF | 58 | ggc [symbol] tttgatattattaaaaaaattgcggaaagcttt |
| Aurein 3 | 39 | GL [symbol] DIIKKIAESF | 59 | ggcctg [symbol] gatattattaaaaaaattgcggaaagcttt |
| Aurein 4 | 40 | GLF [symbol] IIKKIAESF | 60 | ggcctgttt [symbol] attattaaaaaaattgcggaaagcttt |
| Aurein 5 | 41 | GLFD [symbol] IKKIAESF | 61 | ggcctgtttgat [symbol] attaaaaaaattgcggaaagcttt |
| Aurein 6 | 42 | GLFDI [symbol] KKIAESF | 62 | ggcctgtttgatatt [symbol] aaaaaaattgcggaaagcttt |
| Aurein 7 | 43 | GLFDII [symbol] KIAESF | 63 | ggcctgtttgatattatt [symbol] aaaattgcggaaagcttt |
| Aurein 8 | 44 | GLFDIIK [symbol] IAESF | 64 | ggcctgtttgatattattaaa [symbol] attgcggaaagcttt |
| Aurein 9 | 45 | GLFDIIKK [symbol] AESF | 65 | ggcctgtttgatattattaaaaaa [symbol] gcggaaagcttt |
| Aurein 10 | 46 | GLFDIIKKIA [symbol] SF | 66 | ggcctgtttgatattattaaaaaaattgcg [symbol] agcttt |
| Aurein 11 | 47 | GLFDIIKKIAE [symbol] F | 67 | ggcctgtttgatattattaaaaaaattgcggaa [symbol] ttt |
| Aurein 12 | 48 | GLFDIIKKIAES [symbol] | 68 | ggcctgtttgatattattaaaaaaattgcggaaagc [symbol] |
| Aurein 7.His | 49 | GLFDIIHKIAESF | 69 | ggcctgtttgatattattcacaaaattgcggaaagcttt |
| Aurein 8.His | 50 | GLFDIIKHIAESF | 70 | ggcctgtttgatattattaaacacattgcggaaagcttt |
| Aurein 10.His | 51 | GLFDIIKKIAHSF | 71 | ggcctgtttgatattattaaaaaaattgcgcacagcttt |
| Aurein 7.Arg | 52 | GLFDIIRKIAESF | 72 | ggcctgtttgatattattcgcaaaattgcggaaagcttt |
| Aurein 8.Arg | 53 | GLFDIIKRIAESF | 73 | ggcctgtttgatattattaaacgcattgcggaaagcttt |
| Aurein 10.Arg | 54 | GLFDIIKKIARSF | 74 | ggcctgtttgatattattaaaaaaattgcgcgcagcttt |
| Aurein 10.Asp | 55 | GLFDIIKKIADSF | 75 | ggcctgtttgatattattaaaaaaattgcggacagcttt |

Protein Sequences

+36 GFP-Cre: (SEQ ID NO: 76)
MGGGSGGSGGSGGSGGSGGSGGSGGSGGSSKGERLFRGKVPILVELKGDV
NGHKFSVRGKGKGDATRGKLTLKFICTTGKLPVPWPTLVTTLTYGVQCFS
RYPKHMKRHDFFKSAMPKGYVQERTISFKKDGKYKTRAEVKFEGRTLVNR
IKLKGRDFKEKGNILGHKLRYNFNSHKVYITADKRKNGIKAKFKIRHNVK
DGSVQLADHYQQNTPIGRGPVLLPRNHYLSTRSKLSKDPKEKRDHMVLLE
FVTAAGIKHGRDERYKTGGSGGSGGSGGSGGSGGSGGSGGSGGTASNLLT
VHQNLPALPVDATSDEVRKNLMDMFRDRQAFSEHTWKMLLSVCRSWAAWC
KLNNRKWFPAEPEDVRDYLLYLQARGLAVKTIQQHLGQLNMLHRRSGLPR
PSDSNAVSLVMRRIRKENVDAGERAKQALAFERTDFDQVRSLMENSDRCQ
DIRNLAFLGIAYNTLLRIAEIARIRVKDISRTDGGRMLIHIGRTKTLVST
AGVEKALSLGVTKLVERWISVSGVADDPNNYLFCRVRKNGVAAPSATSQL
STRALEGIFEATHRLIYGAKDDSGQRYLAWSGHSARVGAARDMARAGVSI
PEIMQAGGWTNVNIVMNYIRNLDSETGAMVRLLEDGDGGS
Aurein 1.2-+36 GFP-Cre: (SEQ ID NO: 77)
MGLFDIIKKIAESFASGGSGGSGGSGGSGGSGGSGGSGGSGGSSKGERLF
RGKVPILVELKGDVNGHKFSVRGKGKGDATRGKLTLKFICTTGKLPVPWP
TLVTTLTYGVQCFSRYPKHMKRHDFFKSAMPKGYVQERTISFKKDGKYKT
RAEVKFEGRTLVNRIKLKGRDFKEKGNILGHKLRYNFNSHKVYITADKRK
NGIKAKFKIRHNVKDGSVQLADHYQQNTPIGRGPVLLPRNHYLSTRSKLS
KDPKEKRDHMVLLEFVTAAGIKHGRDERYKTGGSGGSGGSGGSGGSGGSG
GSGGSGGTASNLLTVHQNLPALPVDATSDEVRKNLMDMFRDRQAFSEHTW
KMLLSVCRSWAAWCKLNNRKWFPAEPEDVRDYLLYLQARGLAVKTIQQHL
GQLNMLHRRSGLPRPSDSNAVSLVMRRIRKENVDAGERAKQALAFERTDF
DQVRSLMENSDRCQDIRNLAFLGIAYNTLLRIAEIARIRVKDISRTDGGR
MLIHIGRTKTLVSTAGVEKALSLGVTKLVERWISVSGVADDPNNYLFCRV
RKNGVAAPSATSQLSTRALEGIFEATHRLIYGAKDDSGQRYLAWSGHSAR
VGAARDMARAGVSIPEIMQAGGWTNVNIVMNYIRNLDSETGAMVRLLEDG
DGGS
U-+36 GFP-Cre: (SEQ ID NO: 78)
MGLFDIIKKVASVIGGLASGGSGGSGGSGGSGGSGGSGGSGGSGGSSKGE
RLFRGKVPILVELKGDVNGHKFSVRGKGKGDATRGKLTLKFICTTGKLPV
PWPTLVTTLTYGVQCFSRYPKHMKRHDFFKSAMPKGYVQERTISFKKDGK
YKTRAEVKFEGRTLVNRIKLKGRDFKEKGNILGHKLRYNFNSHKVYITAD
KRKNGIKAKFKIRHNVKDGSVQLADHYQQNTPIGRGPVLLPRNHYLSTRS
KLSKDPKEKRDHMVLLEFVTAAGIKHGRDERYKTGGSGGSGGSGGSGGSG
GSGGSGGSGGTASNLLTVHQNLPALPVDATSDEVRKNLMDMFRDRQAFSE
HTWKMLLSVCRSWAAWCKLNNRKWFPAEPEDVRDYLLYLQARGLAVKTIQ
QHLGQLNMLHRRSGLPRPSDSNAVSLVMRRIRKENVDAGERAKQALAFER
TDFDQVRSLMENSDRCQDIRNLAFLGIAYNTLLRIAEIARIRVKDISRTD
GGRMLIHIGRTKTLVSTAGVEKALSLGVTKLVERWISVSGVADDPNNYLF
CRVRKNGVAAPSATSQLSTRALEGIFEATHRLIYGAKDDSGQRYLAWSGH
SARVGAARDMARAGVSIPEIMQAGGWTNVNIVMNYIRNLDSETGAMVRLL
EDGDGGS
His-TEV-U-+36 GFP-Cre: (SEQ ID NO: 79)
MHHHHHHENLYFQGLFDIIKKVASVIGGLASGGSGGSGGSGGSGGSGGSG
GSGGSGGSSKGERLFRGKVPILVELKGDVNGHKFSVRGKGKGDATRGKLT
LKFICTTGKLPVPWPTLVTTLTYGVQCFSRYPKHMKRHDFFKSAMPKGYV
QERTISFKKDGKYKTRAEVKFEGRTLVNRIKLKGRDFKEKGNILGHKLRY
NFNSHKVYITADKRKNGIKAKFKIRHNVKDGSVQLADHYQQNTPIGRGPV
LLPRNHYLSTRSKLSKDPKEKRDHMVLLEFVTAAGIKHGRDERYKTGGSG
GSGGSGGSGGSGGSGGSGGSGGTASNLLTVHQNLPALPVDATSDEVRKNL
MDMFRDRQAFSEHTWKMLLSVCRSWAAWCKLNNRKWFPAEPEDVRDYLLY
LQARGLAVKTIQQHLGQLNMLHRRSGLPRPSDSNAVSLVMRRIRKENVDA
GERAKQALAFERTDFDQVRSLMENSDRCQDIRNLAFLGIAYNTLLRIAEI
ARIRVKDISRTDGGRMLIHIGRTKTLVSTAGVEKALSLGVTKLVERWISV
SGVADDPNNYLFCRVRKNGVAAPSATSQLSTRALEGIFEATHRLIYGAKD
DSGQRYLAWSGHSARVGAARDMARAGVSIPEIMQAGGWTNVNIVMNYIRN
LDSETGAMVRLLEDGDGGS
+36 GFP-BirA: (SEQ ID NO: 80)
MGGGSGGSGGSGGSGGSGGSGGSGGSGGSSKGERLFRGKVPILVELKGDV
NGHKFSVRGKGKGDATRGKLTLKFICTTGKLPVPWPTLVTTLTYGVQCFS
RYPKHMKRHDFFKSAMPKGYVQERTISFKKDGKYKTRAEVKFEGRTLVNR
IKLKGRDFKEKGNILGHKLRYNFNSHKVYITADKRKNGIKAKFKIRHNVK
DGSVQLADHYQQNTPIGRGPVLLPRNHYLSTRSKLSKDPKEKRDHMVLLE
FVTAAGIKHGRDERYKTGGSGGSGGSGGSGGSGGSGGSGGSGGSKDNTVP
LKLIALLANGEFHSGEQLGETLGMSRAAINKHIQTLRDWGVDVFTVPGKG
YSLPEPIQLLNAKQILGQLDGGSVAVLPVIDSTNQYLLDRIGELKSGDAC
IAEYQQAGRGRRGRKWFSPFGANLYLSMFWRLEQGPAAAIGLSLVIGIVM
AEVLRKLGADKVRVKWPNDLYLQDRKLAGILVELTGKTGDAAQIVIGAGI
NMAMRRVEESVVNQGWITLQEAGINLDRNTLAAMLIRELRAALELFEQEG
LAPYLSRWEKLDNFINRPVKLIIGDKEIFGISRGIDKQGALLLEQDGIIK
PWMGGEISLRSAEKGGSHHHHHH
Aurein 1.2-+36 GFP-BirA: (SEQ ID NO: 81)
MGLFDIIKKIAESFASGGSGGSGGSGGSGGSGGSGGSGGSGGSSKGERLF
RGKVPILVELKGDVNGHKFSVRGKGKGDATRGKLTLKFICTTGKLPVPWP
TLVTTLTYGVQCFSRYPKHMKRHDFFKSAMPKGYVQERTISFKKDGKYKT
RAEVKFEGRTLVNRIKLKGRDFKEKGNILGHKLRYNFNSHKVYITADKRK
NGIKAKFKIRHNVKDGSVQLADHYQQNTPIGRGPVLLPRNHYLSTRSKLS
KDPKEKRDHMVLLEFVTAAGIKHGRDERYKTGGSGGSGGSGGSGGSGGSG
GSGGSGGSKDNTVPLKLIALLANGEFHSGEQLGETLGMSRAAINKHIQTL
RDWGVDVFTVPGKGYSLPEPIQLLNAKQILGQLDGGSVAVLPVIDSTNQY
LLDRIGELKSGDACIAEYQQAGRGRRGRKWFSPFGANLYLSMFWRLEQGP
AAAIGLSLVIGIVMAEVLRKLGADKVRVKWPNDLYLQDRKLAGILVELTG
KTGDAAQIVIGAGINMAMRRVEESVVNQGWITLQEAGINLDRNTLAAMLI
RELRAALELFEQEGLAPYLSRWEKLDNFINRPVKLIIGDKEIFGISRGID
KQGALLLEQDGIIKPWMGGEISLRSAEKGGSHHHHHH
U-+36 GFP-BirA: (SEQ ID NO: 82)
MGLFDIIKKVASVIGGLASGGSGGSGGSGGSGGSGGSGGSGGSGGSSKGE
RLFRGKVPILVELKGDVNGHKFSVRGKGKGDATRGKLTLKFICTTGKLPV
PWPTLVTTLTYGVQCFSRYPKHMKRHDFFKSAMPKGYVQERTISFKKDGK
YKTRAEVKFEGRTLVNRIKLKGRDFKEKGNILGHKLRYNFNSHKVYITAD
KRKNGIKAKFKIRHNVKDGSVQLADHYQQNTPIGRGPVLLPRNHYLSTRS
KLSKDPKEKRDHMVLLEFVTAAGIKHGRDERYKTGGSGGSGGSGGSGGSG
GSGGSGGSGGSKDNTVPLKLIALLANGEFHSGEQLGETLGMSRAAINKHI
QTLRDWGVDVFTVPGKGYSLPEPIQLLNAKQILGQLDGGSVAVLPVIDST
NQYLLDRIGELKSGDACIAEYQQAGRGRRGRKWFSPFGANLYLSMFWRLE
QGPAAAIGLSLVIGIVMAEVLRKLGADKVRVKWPNDLYLQDRKLAGILVE
LTGKTGDAAQIVIGAGINMAMRRVEESVVNQGWITLQEAGINLDRNTLAA
MLIRELRAALELFEQEGLAPYLSRWEKLDNFINRPVKLIIGDKEIFGISR
GIDKQGALLLEQDGIIKPWMGGEISLRSAEKGGSHHHHHH
+36 GFP-LPETG: (SEQ ID NO: 83)
MGGGSGGSGGSGGSGGSGGSGGSGGSGGSSKGERLFRGKVPILVELKGDV
NGHKFSVRGKGKGDATRGKLTLKFICTTGKLPVPWPTLVTTLTYGVQCFS
RYPKHMKRHDFFKSAMPKGYVQERTISFKKDGKYKTRAEVKFEGRTLVNR
IKLKGRDFKEKGNILGHKLRYNFNSHKVYITADKRKNGIKAKFKIRHNVK
DGSVQLADHYQQNTPIGRGPVLLPRNHYLSTRSKLSKDPKEKRDHMVLLE
FVTAAGIKHGRDERYKTGGSLPETGHHHHHH
His-TEV-Aurein 1.2-+36 GFP-LPETG: (SEQ ID NO: 84)
MHHHHHHENLYFQGLFDIIKKIAESFASGGSGGSGGSGGSGGSGGSGGSG
GSGGSSKGERLFRGKVPILVELKGDVNGHKFSVRGKGKGDATRGKLTLKF
ICTTGKLPVPWPTLVTTLTYGVQCFSRYPKHMKRHDFFKSAMPKGYVQER
TISFKKDGKYKTRAEVKFEGRTLVNRIKLKGRDFKEKGNILGHKLRYNFN
SHKVYITADKRKNGIKAKFKIRHNVKDGSVQLADHYQQNTPIGRGPVLLP
RNHYLSTRSKLSKDPKEKRDHMVLLEFVTAAGIKHGRDERYKTGGSLPET
GHHHHHH
+36 GFP-Cys: (SEQ ID NO: 85)
MGGGSGGSGGSGGSGGSGGSGGSGGSGGSSKGERLFRGKVPILVELKGDV
NGHKFSVRGKGKGDATRGKLTLKFICTTGKLPVPWPTLVTTLTYGVQCFS
RYPKHMKRHDFFKSAMPKGYVQERTISFKKDGKYKTRAEVKFEGRTLVNR
IKLKGRDFKEKGNILGHKLRYNFNSHKVYITADKRKNGIKAKFKIRHNVK
DGSVQLADHYQQNTPIGRGPVLLPRNHYLSTRSKLSKDPKEKRDHMVLLE
FVTAAGIKHGRDERYKTGGSGCGGSHHHHHH
Aurein 1.2-+36 GFP-Cys: (SEQ ID NO: 86)
MGLFDIIKKIAESFASGGSGGSGGSGGSGGSGGSGGSGGSGGSSKGERLF
RGKVPILVELKGDVNGHKFSVRGKGKGDATRGKLTLKFICTTGKLPVPWP
TLVTTLTYGVQCFSRYPKHMKRHDFFKSAMPKGYVQERTISFKKDGKYKT
RAEVKFEGRTLVNRIKLKGRDFKEKGNILGHKLRYNFNSHKVYITADKRK
NGIKAKFKIRHNVKDGSVQLADHYQQNTPIGRGPVLLPRNHYLSTRSKLS
KDPKEKRDHMVLLEFVTAAGIKHGRDERYKTGGSGCGGSHHHHHH
U-+36 GFP-Cys: (SEQ ID NO: 87)
MGLFDIIKKVASVIGGLASGGSGGSGGSGGSGGSGGSGGSGGSGGSSKGE
RLFRGKVPILVELKGDVNGHKFSVRGKGKGDATRGKLTLKFICTTGKLPV
PWPTLVTTLTYGVQCFSRYPKHMKRHDFFKSAMPKGYVQERTISFKKDGK
YKTRAEVKFEGRTLVNRIKLKGRDFKEKGNILGHKLRYNFNSHKVYITAD
KRKNGIKAKFKIRHNVKDGSVQLADHYQQNTPIGRGPVLLPRNHYLSTRS
KLSKDPKEKRDHMVLLEFVTAAGIKHGRDERYKTGGSGCGGSHHHHHH
AP-mCherry: (SEQ ID NO: 88)
MGLNDIFEAQKIEWHEGGSVSKGEEDNMAIIKEFMRFKVHMEGSVNGHEF
EIEGEGEGRPYEGTQTAKLKVTKGGPLPFAWDILSPQFMYGSKAYVKHPA
DIPDYLKLSFPEGFKWERVMNFEDGGVVTVTQDSSLQDGEFIYKVKLRGT
NFPSDGPVMQKKTMGWEASSERMYPEDGALKGEIKQRLKLKDGGHYDAEV
KTTYKAKKPVQLPGAYNVNIKLDITSHNEDYTIVEQYERAEGRHSTGGMD
ELYK

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A protein comprising a peptide sequence that is at least 90% identical to any one of the following amino acid sequences:

| SEQ ID NO: | Amino Acid Sequence |
|---|---|
| 1 | FLFPLITSFLSKVL |
| 2 | FISAIASMLGKFL |
| 3 | GWFDVVKHIASAV |
| 4 | FFGSVLKLIPKIL |
| 5 | GLFDIIKKIAESF |
| 6 | HGVSGHGQHGVHG |
| 7 | FLPLIGRVLSGIL |
| 8 | GLFDIIKKIAESI |
| 9 | GLLDIVKKVVGAFGSL |
| 10 | GLFDIVKKVVGALGSL |
| 11 | GLFDIVKKVVGAIGSL |
| 12 | GLFDIVKKVVGTLAGL |
| 13 | GLFDIVKKVVGAFGSL |
| 14 | GLFDIAKKVIGVIGSL |
| 15 | GLFDIVKKIAGHIAGSI |
| 16 | GLFDIVKKIAGHIASSI |
| 17 | GLFDIVKKIAGHIVSSI |
| 18 | FVQWFSKFLGRIL |
| 19 | GLFDVIKKVASVIGGL |
| 20 | GLFDIIKKVASVVGGL |
| 21 | GLFDIIKKVASVIGGL |
| 22 | VWPLGLVICKALKIC |
| 23 | NFLGTLVNLAKKIL |
| 24 | FLPLIGKILGTIL |
| 25 | FLPIIAKVLSGLL |
| 26 | FLPIVGKLLSGLL |
| 27 | FLSSIGKILGNLL |
| 28 | FLSGIVGMLGKLF |
| 29 | TPFKLSLHL |
| 30 | GILDAIKAIAKAAG |
| 31 | LFDIIKKIAESF |
| 32 | LFDIIKKIAESGFLFDIIKKIAESF |
| 33 | GLLNGLALRLGKRALKKIIKRLCR |
| 34 | GHHHHHHHHHHHHH |
| 35 | FKCRRWQWRM |
| 36 | KTCENLADTY |
| 37 | ALFDIIKKIAESF |
| 38 | GAFDIIKKIAESF |
| 49 | GLADIIKKIAESF |
| 40 | GLFAIIKKIAESF |
| 41 | GLFDAIKKIAESF |
| 42 | GLFDIAKKIAESF |
| 43 | GLFDIIAKIAESF |
| 47 | GLFDIIKAIAESF |
| 45 | GLFDIIKKAAESF |
| 46 | GLFDIIKKIAASF |
| 47 | GLFDIIKKIAEAF |
| 48 | GLFDIIKKIAESA |
| 59 | GLFDIIHKIAESF |
| 50 | GLFDIIKHIAESF |
| 51 | GLFDIIKKIAHSF |
| 52 | GLFDIIRKIAESF |
| 53 | GLFDIIKRIAESF |
| 54 | GLFDIIKKIARSF |
| 55 | GLFDIIKKIADSF |

fused to a protein for delivery to a cell.

2. The protein of claim 1, wherein the peptide sequence is at least 95% identical to any one of the amino acid sequences set forth in SEQ ID NOs: 1-55.

3-4. (canceled)

5. The protein of claim 1, wherein the peptide sequence is identical to any one of the amino acid sequences set forth in SEQ ID NOs: 1-55.

6. The protein of claim 1, wherein the peptide sequence comprises any one of the amino acid sequences set forth in SEQ ID NOs: 1-55; optionally with 1, 2, 3, 4, or 5 amino acid additions, deletions, substitutions, mutations, or any combination thereof.

7. The protein of claim 1, wherein the peptide sequence is at least 90% identical to the amino acid sequence set forth in SEQ ID NO: 5.

8. The protein of claim 1, wherein the protein is a therapeutic protein.

9. The protein of claim 1, wherein the protein is an enzyme.

10. The protein of claim 1, wherein the protein is selected from the group consisting of Cas9 proteins, nucleases, nickases, methylases, recombinases, deaminases, DNA methyltransferases, histone-modifying enzymes, and transcription factors.

11. The protein of claim 1, wherein the protein is a cationic protein.

12. The protein of claim 11, wherein the protein is a supercharged protein, wherein the supercharged protein has an overall greater net charge than its corresponding wild-type protein.

13-16. (canceled)

17. The protein of claim 1, further comprising a supercharged protein, wherein the supercharged protein has an overall greater net charge than its corresponding wild-type protein.

18. The protein of claim 1, further comprising a therapeutic protein.

19-20. (canceled)

21. A conjugate comprising a peptide sequence that is at least 90% identical to any one of the amino acid sequences set forth in SEQ ID NOs: 1-55, conjugated to a small molecule or nucleic acid for delivery to a cell.

22-35. (canceled)

36. A nucleic acid for encoding a protein of claim 1.

37. An expression vector for a protein of claim 1.

38. A pharmaceutical composition comprising:
a protein or conjugate of claim 1; and
a pharmaceutically acceptable excipient.

39. A method comprising administering the protein of claim 1 to a subject.

40-42. (canceled)

43. A peptide of the structure: [first peptide]-[first sortase recognition motif], wherein
the first peptide comprises an amino acid sequence that is at least 90% identical to any one of the amino acid sequences set forth in SEQ ID NOs: 1-55; and
the first sortase recognition motif is a peptide.

44-51. (canceled)

52. A method of preparing a fusion protein of claim 1, the method comprising contacting:
(1) a peptide of claim 43 of the structure: [first peptide]-[first sortase recognition motif]; with
(2) a substrate of the structure: [second sortase recognition motif]-[second agent], wherein the second agent comprises one or more agents selected from the group consisting of of proteins, peptides, nucleic acids, and small molecules; and
(3) a sortase;
under conditions suitable for the sortase to catalyze a transpeptidation reaction.

53-73. (canceled)

74. A peptide comprising a peptide sequence that is at least 90% identical to any one of the following amino acid sequences:

| SEQ ID NO: | Amino Acid Sequence |
|---|---|
| 37 | ALFDIIKKIAESF |
| 38 | GAFDIIKKIAESF |
| 49 | GLADIIKKIAESF |
| 40 | GLFAIIKKIAESF |
| 41 | GLFDAIKKIAESF |
| 42 | GLFDIAKKIAESF |
| 43 | GLFDIIAKIAESF |
| 47 | GLFDIIKAIAESF |
| 45 | GLFDIIKKAAESF |
| 46 | GLFDIIKKIAASF |
| 47 | GLFDIIKKIAEAF |
| 48 | GLFDIIKKIAESA |
| 59 | GLFDIIHKIAESF |
| 50 | GLFDIIKHIAESF |
| 51 | GLFDIIKKIAHSF |
| 52 | GLFDIIRKIAESF |
| 53 | GLFDIIKRIAESF |
| 54 | GLFDIIKKIARSF |
| 55 | GLFDIIKKIADSF |

75-79. (canceled)
